SEQUENCE LISTING (53 sequences)

SEQ ID NO: 1 - protein, 9 aa, synthetic construct
  REGION 1..9: note = Synthetic
FPLLAECLF

SEQ ID NO: 2 - protein, 15 aa, synthetic construct
  REGION 1..15: note = Synthetic
LHPRDLLGSPITAFG

SEQ ID NO: 3 - protein, 19 aa, synthetic construct
  REGION 1..19: note = Synthetic
GLPPASRRRPVVGEFLWDD

SEQ ID NO: 4 - protein, 15 aa, synthetic construct
  REGION 1..15: note = Synthetic
SKLRSLTPISTSSLC

SEQ ID NO: 5 - protein, 15 aa, synthetic construct
  REGION 1..15: note = Synthetic
LYHGNNPPKKFGVIC

SEQ ID NO: 6 - protein, 15 aa, synthetic construct
  REGION 1..15: note = Synthetic
LDFWHHEVNTLITKA

SEQ ID NO: 7 - protein, 9 aa, synthetic construct
  REGION 1..9: note = Synthetic
TFPLLAECL

SEQ ID NO: 8 - protein, 19 aa, synthetic construct
  REGION 1..19: note = Synthetic
LSKGLHPRDLLGSPITAFG

SEQ ID NO: 9 - protein, 9 aa, synthetic construct
  REGION 1..9: note = Synthetic
RRRPVVGEF

SEQ ID NO: 10 - protein, 9 aa, synthetic construct
  REGION 1..9: note = Synthetic
RRPVVGEFL

SEQ ID NO: 11 - protein, 9 aa, synthetic construct
  REGION 1..9: note = Synthetic
PLVIKLRLR

SEQ ID NO: 12 - protein, 9 aa, synthetic construct
  REGION 1..9: note = Synthetic
LVIKLRLRS

SEQ ID NO: 13 - protein, 9 aa, synthetic construct
  REGION 1..9: note = Synthetic
VIKLRLRSV

SEQ ID NO: 14 - protein, 15 aa, synthetic construct
  REGION 1..15: note = Synthetic
LSKGLHPRDLLGSPI

SEQ ID NO: 15 - protein, 15 aa, synthetic construct
  REGION 1..15: note = Synthetic
DMVPLVIKLRLRSVT

SEQ ID NO: 16-19 - skipped (4 entries)

SEQ ID NO: 20 - protein, 15 aa, synthetic construct
  REGION 1..15: note = Synthetic
EVLFKFLTTEFGKSV

SEQ ID NO: 21 - protein, 15 aa, synthetic construct
  REGION 1..15: note = Synthetic
KFLTTEFGKSVINVD

SEQ ID NO: 22 - protein, 15 aa, synthetic construct
  REGION 1..15: note = Synthetic
KQSLGWVLTSAALLI

SEQ ID NO: 23 - protein, 15 aa, synthetic construct
  REGION 1..15: note = Synthetic
GWVLTSAALLIALVL

SEQ ID NO: 24 - protein, 15 aa, synthetic construct
  REGION 1..15: note = Synthetic
GLPPASRRRPVVGEF

SEQ ID NO: 25 - protein, 15 aa, synthetic construct
  REGION 1..15: note = Synthetic
ASRRRPVVGEFLWDD

SEQ ID NO: 26 - protein, 15 aa, synthetic construct
  REGION 1..15: note = Synthetic
GRLTFPLLAECLFRA

SEQ ID NO: 27 - protein, 15 aa, synthetic construct
  REGION 1..15: note = Synthetic
FPLLAECLFRAGRRD

SEQ ID NO: 28 - protein, 15 aa, synthetic construct
  REGION 1..15: note = Synthetic
IGSRSTPQTFLHWVY

SEQ ID NO: 29 - protein, 15 aa, synthetic construct
  REGION 1..15: note = Synthetic
STPQTFLHWVYCMEN

SEQ ID NO: 30 - protein, 188 aa, synthetic construct
  REGION 1..188: note = Synthetic
MATYEVLCEVARKLGTDDREVVLFLLNVFIPQPTLAQLIGALRALKEEGRLTFPLLAECLFRAGRRDLLRDLLHLDPRFLERHLAGTMSYFSPYQLTVLHVDGELCARDIRSLIFLSKDTIGSRSTPQTFLHWVYCMENLDLLGPTDVDALMSMLRSLSRVDLQRQVQTLMGLHLSGPSHSQHYRHTP

SEQ ID NO: 31 - protein, 204 aa, synthetic construct
  REGION 1..204: note = Synthetic
MCWFKLWSLLLVGSLLVSGTRGKLPDAPEFEKDLLIQRLNWMLWVIDECFRDLCYRTGICKGILEPAAIFHLKLPAINDTDHCGLIGFNETSCLKKLADGFFEFEVLFKFLTTEFGKSVINVDVMELLTKTLGWDIQEELNKLTKTHYSPPKFDRGLLGRLQGLKYWVRHFASFYVLSAMEKFAGQAVRVLDSIPDVTPDVHDK

SEQ ID NO: 32 - protein, 257 aa, synthetic construct
  REGION 1..257: note = Synthetic
MATANNPPSGLLDPTLCEDRIFYNILEIEPRFLTSDSVFGTFQQSLTSHMRKLLGTWMFSVCQEYNLEPNVVALALNLLDRLLLIKQVSKEHFQKTGSACLLVASKLRSLTPISTSSLCYAAADSFSRQELIDQEKELLEKLAWRTEAVLATDVTSFLLLKLLGGSQHLDFWHHEVNTLITKALVDPKTGSLPASIISAAGCALLVPANVIPQDTHSGGVVPQLASILGCDVSVLQAAVEQILTSVSDFDLRILDSY

SEQ ID NO: 33 - protein, 342 aa, synthetic construct
  REGION 1..342: note = Synthetic
MAAEDFLTIFLDDDESWNETLNMSGYDYSGNFSLEVSVCEMTTVVPYTWNVGILSLIFLINVLGNGLVTYIFCKHRSRAGAIDILLLGICLNSLCLSISLLAEVLMFLFPNIISTGLCRLEIFFYYLYVYLDIFSVVCVSLVRYLLVAYSTRSWPKKQSLGWVLTSAALLIALVLSGDACRHRSRVVDPVSKQAMCYENAGNMTADWRLHVRTVSVTAGFLLPLALLILFYALTWCVVRRTKLQARRKVRGVIVAVVLLFFVFCFPYHVLNLLDTLLRRRWIRDSCYTRGLINVGLAVTSLLQALYSAVVPLIYSCLGSLFRQRMYGLFQSLRQSFMSGATT

SEQ ID NO: 34 - protein, 566 aa, synthetic construct
  REGION 1..566: note = Synthetic
MAGRRLTWISEFIVGALDSDKYPLVKWLDRSTGTFLAPAARNDVIPLDSLQFFIDFKRECLSKGLHPRDLLGSPITAFGKICTTSRRLRRLPGEEYEVVQGINCRRWRLLCAEVKECWWCVHARTHLHSGSSLWEILYQHSVRLEKHRRRPRPFVGENSDSSEEDHPAFCDVPVTQTGAESEDSGDEGPSTRHSASGVQPVDDANADSPGSGDEGPSTRHSDSQPPPADETTVHTDNVEDDLTLLDKESACALMYHVGQEMDMLMRAMCDEDLFDLLGIPEDVIATSQPGGDTDASGVVTEGSIAASAVGAGVEDVYLAGALEAQNVAGEYVLEISDEEVDDGAGLPPASRRRPVVGEFLWDDGPRRHERPTTRRIRHRKLRSAYYRVARPPVMITDRLGVEVFYFGRPAMSLEVERKVFILCSQNPLADISHSCLHSRKGLRVLLPKPDDNNTGPGDVNLLAAVLRSFASGLVIVSLRSGIYVKNLCKSTVLYHGNNPPKKFGVICGLSSRAVLDVFNVAQYRIQGHEHIKKTTVFIGGDPTSAEQFDMVPLVIKLRLRSVTCDD

SEQ ID NO: 35 - protein, 15 aa, Homo sapiens
  REGION 1..15: note = misc_feature - Human complement v-CYCLIN SKL12 peptide
SKLKETSPLTAEKLC

SEQ ID NO: 36 - protein, 9 aa, synthetic construct
  REGION 1..9: note = Synthetic
CLFRAGRRD

SEQ ID NO: 37 - protein, 9 aa, synthetic construct
  REGION 1..9: note = Synthetic
ECLFRAGRR

SEQ ID NO: 38 - protein, 9 aa, synthetic construct
  REGION 1..9: note = Synthetic
AECLFRAGR

SEQ ID NO: 39 - protein, 9 aa, synthetic construct
  REGION 1..9: note = Synthetic
LAECLFRAG

SEQ ID NO: 40 - protein, 9 aa, synthetic construct
  REGION 1..9: note = Synthetic
LLAECLFRA

SEQ ID NO: 41 - protein, 9 aa, synthetic construct
  REGION 1..9: note = Synthetic
PLLAECLFR

SEQ ID NO: 42 - protein, 9 aa, synthetic construct
  REGION 1..9: note = Synthetic
LTFPLLAEC

SEQ ID NO: 43 - protein, 9 aa, synthetic construct
  REGION 1..9: note = Synthetic
RLTFPLLAE

SEQ ID NO: 44 - protein, 9 aa, synthetic construct
  REGION 1..9: note = Synthetic
GRLTFPLLA

SEQ ID NO: 45 - protein, 9 aa, synthetic construct
  REGION 1..9: note = Synthetic
RRRPVVGEL

SEQ ID NO: 46 - protein, 9 aa, synthetic construct
  REGION 1..9: note = Synthetic
FPLLAECLL

SEQ ID NO: 47 - protein, 15 aa, Homo sapiens
  REGION 1..15: note = misc_feature - human IL6
EVYLEYLQNRFESSE

SEQ ID NO: 48 - protein, 9 aa, synthetic construct
  REGION 1..9: note = Synthetic
LFKFLTTEF

SEQ ID NO: 49 - protein, 9 aa, synthetic construct
  REGION 1..9: note = Synthetic
TTEFGKSVI

SEQ ID NO: 50 - protein, 15 aa, Homo sapiens
  REGION 1..15: note = misc_feature - human GPCR
HNYTIWSVLVAIWTV

SEQ ID NO: 51 - protein, 9 aa, synthetic construct
  REGION 1..9: note = Synthetic
SLGWVLTSA

SEQ ID NO: 52 - protein, 9 aa, synthetic construct
  REGION 1..9: note = Synthetic
TSAALLIAL

SEQ ID NO: 53 - protein, 9 aa, synthetic construct
  REGION 1..9: note = Synthetic
SAALLIALV

(19) United States
(12) Patent Application Publication
(10) Pub. No.: US 2024/0000936 A1
(43) Pub. Date: Jan. 4, 2024

(54) KSHV ONCOPROTEIN ANTIGENS AND EPITOPES FOR EXPANDING ANTIGEN-SPECIFIC T CELLS

(71) Applicants: Children's National Medical Center, Washington, DC (US); Fred Hutchinson Cancer Center, Seattle, WA (US)

(72) Inventors: Catherine Mary Bollard, Washington, DC (US); Danielle Sohai, Washington, DC (US); Conrad Russell Y. Cruz, Washington, DC (US); Thomas Uldrick, Seattle, WA (US)

(21) Appl. No.: 18/310,649

(22) Filed: May 2, 2023

Related U.S. Application Data
(60) Provisional application No. 63/337,814, filed on May 3, 2022.

(51) Int. Cl. A61K 39/00; C12N 5/0783; A61P 31/22; A61P 35/00

(52) U.S. Cl. CPC A61K 39/4611; C12N 5/0636; A61K 39/464838; A61P 31/22; A61P 35/00; A61K 39/464449; A61K 39/46444; A61K 39/464402; A61K 39/464452; C12N 2502/1121; C12N 2501/2302; C12N 2501/2307; C12N 2501/2315; C12N 2501/2321; A61K 2239/57; A61K 2239/26

(57) ABSTRACT

The invention described herein provide Kaposi Sarcoma-Associated Herpesvirus (KSHV) oncoprotein antigens and epitopes for expanding antigen-specific T cells. Such expanded T cells are useful for, e.g., in allogeneic or “off-the-shelf” adoptive T cell therapy.

### REFERENCE TO RELATED APPLICATION

This application claims priority to and the benefit of the filing date of U.S. Provisional Patent Application No. 63/337,814, filed on May 3, 2022, the entire contents of which are incorporated herein by reference.

### REFERENCE TO SEQUENCE LISTING

The instant application contains a Sequence Listing which has been submitted electronically in XML format and is hereby incorporated by reference in its entirety. Said XML copy, created on May 1, 2023, is named 135372-02402.XML and is 63,852 bytes in size.

### BACKGROUND OF THE INVENTION

Kaposi sarcoma-associated herpesvirus (KSHV), or human herpesvirus-8 (HHV8), a gamma-herpesvirus capable of lifelong latent infection, is the etiologic agent of several rare oncogenic diseases, including Kaposi sarcoma (KS), primary effusion lymphoma (PEL), and multicentric Castleman's disease (MCD). Manifestation of these cancers are more common in immune compromised individuals, including those with HIV/AIDS and transplant recipients. Current therapies, including antiviral therapies, radiation, and chemotherapy, are limited in specific clearance of KSHV, have appreciable toxicities, and do not effectively contribute to establishing lasting immunity to prevent relapse.

Immune integrity has been shown to be essential in host control of KSHV infections. This suggests a crucial role for T cell mediated antiviral immunity in controlling KSHV, a postulate further supported by improved KSHV-specific T cell responses in patients receiving highly active antiretroviral therapy (HAART) for the treatment of HIV who exhibit CD4 immune reconstitution (1). Immune compromised patients are at high-risk for developing KSHV-associated cancers due to combinations of primary or persistent latent KSHV infections, co-infections, and disease relapse. It has been shown that patients co-infected with KSHV and other viruses, including HIV, EBV, and/or CMV, exhibit reduced KSHV-specific immune responses and general immune dysfunction, contributing to adverse effects.

Adoptive T cell therapy is a promising approach to augment KSHV-specific immune responses. Infusion of virus-specific T cell (VSTs) products against similar herpesviruses, CMV and EBV, have demonstrated sustained increases in virus-specific T cells in peripheral blood post autologous and allogeneic VST infusion (2). Clinical trials using off-the-shelf VSTs targeting CMV, EBV, and HHV6 in patients with multiple viral infections have demonstrated that the proposed technology is feasible and can be extended to enable targeting of other herpesviruses (3). Moreover, the benefits of allogeneic, or off-the-shelf, T cell therapy allow for rapid treatment as a result of banked VSTs, which limits expenditures of individual VST manufacture. The development of effective third-party VST products necessitates the characterization of immunodominant KSHV-specific T cell epitopes to enhance matching of the donor VST product to the recipient.

Protective T cell responses against KSHV require further investigation and knowledge of the immunodominant KSHV-specific T cell epitopes are limited. In particular, T cell responses targeting KSHV antigens (ORF71 [v-FLIP], ORF72 [v-CYCLIN], ORF74 [v-GPCR], K2 [v-IL6], and ORFK10.5 [vIRF-3]) known to be critical in the tumorigenic process, have not been well defined. In studies assessing host T cell responses against the whole KSHV proteome in seropositive (Roshan et al. 2017; doi.org/10.18632/oncotarget.22683) and seronegative (Nalwoga et al. 2021; doi.org/10.1038/s41467-021-27623-8) individuals, T cell responses against the aforementioned KSHV oncoproteins have weak to no responses.

Identification of dominant epitopes in KSHV oncoproteins may enable adoptive cell therapies to target key drivers of KSHV induced cell growth, proliferation, and cytokine dysregulation observed in the clinical setting. To our knowledge, the ex vivo expansion of KSHV-specific T cell products from virus naïve donors has not been reported. Adoptive cell therapy approaches to treat Kaposi sarcoma-associated herpesvirus are not established.

### SUMMARY OF THE INVENTION

In certain aspects, the present disclosure provides a pharmaceutical composition comprising a Kaposi sarcoma-associated herpesvirus (KSHV)-specific T cell (KST), wherein said KST (1) derives from a naïve T cell that has not been previously exposed to KSHV antigens; (2) has been exposed to, presented with, and/or stimulated by a KSHV antigen or an epitope thereof; and (3) produces a cytokine produced by activated T lymphocytes, upon exposure to said KSHV antigen or said epitope thereof.

In some embodiments, the KST is a human T cell.

In some embodiments, said naïve T cell is isolated from PBMC of a subject naïve to KSHV (e.g., not previously infected by/exposed to KSHV), and optionally further naïve to HIV (e.g., not previously infected by/exposed to HIV).

In some embodiments, said KSHV antigen comprises a KSHV cancer-associated antigen.

In some embodiments, said KSHV antigen comprises vFLIP, vIL-6, vCyclin, vGPCR, vIRF-3, and/or a combination thereof.

In some embodiments, said KST has been exposed to, presented with, and/or stimulated by said KSHV antigen or said epitope thereof through contacting a mature antigen-presenting cell (APC, e.g., a mature dendritic cell) that expresses/presents said KSHV antigen or said epitope thereof.

In some embodiments, said KST is contacted by said APC in the presence of one or more cytokines conductive to antigen presentation and stimulation of T cells.

In some embodiments, said one or more cytokines conductive to antigen presentation and stimulation of T cells comprise IL-2, IL-7, IL-15, IL-21, or any combination thereof.

In some embodiments, said APC is a mature dendritic cell (i) isolated from PBMC as a CD14⁺ immature dendritic cell; (ii) pulsed with said KSHV antigen in the presence of one or more cytokines conductive for dendritic cell maturation (such as GM-CSF, IL-4, IL-6, IL-1β, TNFα, IFNγ, and prostaglandin E2 (PGE2)); and (iii) optionally irradiated (e.g., at 25 Gy).

In some embodiments, said epitope thereof comprises any one or more of the epitopes in FIG. 20, and any one more more of SEQ ID NOs: 1-6 (e.g., any one or more of SEQ ID NOs: 1-29).

In some embodiments, said cytokine produced by activated T lymphocytes comprises IFNγ and/or TNFα.

In some embodiments, the naïve T is a CD14⁻CD45RA⁺ naïve T.

In some aspects the present disclosure provides a method of treating a disease caused by or associated with Kaposi sarcoma-associated herpesvirus (KSHV), in a subject in need thereof, the method comprising administering an effective amount of the pharmaceutical composition of claim 1 to the subject, such that the disease is treated.

In some embodiments, the disease is Kaposi sarcoma (KS), primary effusion lymphoma (PEL), and/or multicentric Castleman's disease (MCD).

In some embodiments, the subject a human.

In some embodiments, the human is immune compromised.

In some embodiments, the human is HIV-positive, such as a human receiving highly active antiretroviral therapy (HAART) for the treatment of HIV who exhibits CD4 immune reconstitution.

In some embodiments, the human is a transplant recipient, and/or is under treatment by an immunosuppressant (such as one or more glucocorticoids, cytostatics, antibodies, and/or drugs acting on immunophilins).

In some embodiments, the subject is co-infected by KSHV and an additional virus (such as HIV, EBV, HHV6, and/or CMV).

In some embodiments, the subject is HLA-matched with the HLA-type of the KST in said pharmaceutical composition.

In other aspects, the present disclosure provides a method of producing a Kaposi sarcoma-associated herpesvirus (KSHV)-specific T cell (KST), the method comprising: contacting a naïve T cell that has not been previously exposed to KSHV antigens with a KSHV antigen or an epitope thereof.

In some embodiments, said KST is a human T cell.

In some embodiments, said naïve T cell is isolated from PBMC of a subject naïve to KSHV (e.g., not previously infected by/exposed to KSHV), and optionally further naïve to HIV (e.g., not previously infected by/exposed to HIV).

In some embodiments, said KSHV antigen comprises a KSHV cancer-associated antigen.

In some embodiments, said KSHV antigen comprises vFLIP, vIL-6, vCyclin, vGPCR, vIRF-3, and/or a combination thereof.

In some embodiments, said naïve T cell is contacted by said KSHV antigen or said epitope thereof through contacting a mature antigen-presenting cell (APC, e.g., a mature dendritic cell) that expresses/presents said KSHV antigen or said epitope thereof.

In some embodiments, said naïve T cell is contacted by said APC in the presence of one or more cytokines conductive to antigen presentation and stimulation of T cells.

In some embodiments, said one or more cytokines conductive to antigen presentation and stimulation of T cells comprise IL-2, IL-7, IL-15, IL-21, or any combination thereof.

In some embodiments, said APC is a mature dendritic cell (i) isolated from PBMC as a CD14⁺ immature dendritic cell; (ii) pulsed with said KSHV antigen in the presence of one or more cytokines conductive for dendritic cell maturation (such as GM-CSF, IL-4, IL-6, IL-1β, TNFα, IFNγ, and prostaglandin E2 (PGE2)); and (iii) optionally irradiated (e.g., at 25 Gy).

In some embodiments, said epitope thereof comprises any one or more of the epitopes in FIG. 20, and any one more more of SEQ ID NOs: 1-6 (e.g., any one or more of SEQ ID NOs: 1-29).

In some embodiments, the method further comprises verifying production of a cytokine produced by activated T lymphocytes (such as IFNγ and/or TNFα) upon exposing said KST to said KSHV antigen or said epitope thereof.

In some embodiments, the mature dendritic cell has an HLA type that matches said KSHV antigen or said epitope thereof based on FIG. 20.

In some embodiments, the naïve T is a CD14⁻CD45RA⁺ naïve T.

Additional experiments are conducted to establish epitope specificities of the expanded KSHV-specific T cell products and to determine HLA-restrictions of the epitopes.

Further, cytotoxicity assays are used to demonstrate that the KSHV-specific T cells have cytolytic ability in vitro.

In addition to the donor-derived products described herein, more KSHV-specific T cell products are expand from seronegative donors.

It should be understood that any one embodiment of the invention described herein, including those described only in the examples or claims, can be combined with any one or more additional embodiments of the invention, unless such combination is improper or expressly disclaimed.

### BRIEF DESCRIPTION OF THE DRAWINGS

FIG. 1-Schematic of KSHV-specific T cell expansion from virus naïve donors. Existing virus-specific T cell protocols have utilized this product generation protocol. Isolation of CD45RA⁺ T cells from bulk peripheral blood mononuclear cells (PBMCs) limited expansion of CD45RA⁻CD45RO⁺ memory T cell expansion. Dendritic cells were pulsed with five pools of KSHV antigens: v-FLIP, v-IL6, v-Cyclin, v-GPCR, and v-IRF3. T cells were co-cultured with antigen-presenting cells over the course of three stimulations.

FIG. 2-Selection of KSHV antigens associated with cancer pathogenesis.

FIG. 3-KSHV-specific T cells (KSTs) were successfully expanded from HIV-negative and KSHV-negative naïve CD45RA-selected T cells. KSHV-specific T cells were expanded from 5 donor PBMCs.

FIG. 4A-Anti-IgG KSHV responses were negative for all donors (OD≤0.15).

FIG. 4B-Cell supernatants assessed for HIV p24 secretion from cultured T cells were negative for all donors (<0 pg/mL HIV-1 p24).

FIG. 5-KST products were specific for 1-2 KSHV antigens as shown by IFN-γ ELISPOT assays.

FIG. 6-KST products were predominantly CD3⁺ (range: 78.4-98.5%) memory T cells with varying expression of CD4 and CD8.

FIGS. 7A-7C—KSHV-specific T cell responses were mapped to 15-mer epitope specificities. Pooled peptide epitope mapping was used to determine epitope specificities. Positive ELISPOT readouts were defined as 2× higher than the actin (negative) control. Cells stimulated with staphylococcal enterotoxin B (SEB) were used as positive controls (not shown). FIG. 7A-K514 products were specific for KSHV v-IRF3 antigens (sequences from top down: SEQ ID NOs: 25, 24, 2 and 14). FIG. 7B-K624 products were specific for KSHV v-FLIP antigens (sequences from top down: SEQ ID NOs: 27 and 26). FIG. 7C-K974 products were specific for v-Cyclin antigens (SEQ ID NO: 4).

FIG. 8-KSHV-specific T cells were polyfunctional in the CD8 compartment. K514 products elicited CD8⁺ T cell responses against v-IRF3 15-mer epitopes. K514 epitope specificities were mapped to 2 pairs of overlapping epitopes: LSKGLHPRDLLGSPI (SEQ ID NO: 14) and LHPRDLLGSPITAFG (SEQ ID NO: 2), and GLPPASRRRPVVGEF (SEQ ID NO: 24) and ASRRRPVVGEFLWDD (SEQ ID NO: 25).

FIGS. 9-K514 products showed broad activation of CD4⁺ T cells against v-IRF3 15-mer epitopes (LSKGLHPRDLLGSPI (SEQ ID NO: 14), LHPRDLLGSPITAFG (SEQ ID NO: 2), GLPPASRRRPVVGEF (SEQ ID NO: 24) and ASRRRPVVGEFLWDD (SEQ ID NO: 25)), and lacked specific responses observed for CD8⁺ T cells.

FIGS. 10A-10B—K624 products showed (FIG. 10A) specific CD8⁺ T cell responses against v-FLIP epitopes. K624 products were mapped to one pair of overlapping epitopes: GRLTFPLLAECLFRA (SEQ ID NO: 26) and FPLLAECLFRAGRRD (SEQ ID NO: 27). (FIG. 10B) CD4⁺ T cells lacked specific responses as demonstrated by background readouts shown in no peptide and actin control conditions like that of peptide-stimulated cells.

FIGS. 11A-11B—K974 products had (FIG. 11A) limited CD8⁺ T cell activation. K974 products demonstrated (FIG. 11B) intracellular CD4⁺ T cell activation. K974 products were mapped to v-CYCLIN SKLRSLTPISTSSLC (SEQ ID NO: 4). KSHV v-IL6, v-Cyclin, and v-GPCR share homology with the human protein variations averaging 28-35% with v-Cyclin sharing ˜33% homology as determined by default protein-protein BLAST search. Here, the data shows the human complement (SKLKETSPLTAEKLC (SEQ ID NO: 35)) of the v-CYCLIN SKL12 peptide would not cross-react with K974 KST products and therefore the KST products may not trigger an autoimmune response in vivo.

FIGS. 12A-12D—Minimal KSHV epitope specificities had high predicted binding affinities. Minimal (9 aa overlapping by 8 aa) peptides were generated from the identified 15-mer epitope specificities. (FIG. 12A) K514 products showed specificity for overlapping minimal epitopes (sequences from top to bottom, SEQ ID NOs, 13, 12, 11, 10 and 9). (FIG. 12B) K624 products showed robust specificity for overlapping epitopes, but specifically the 9mer: FPLLAECLF (SEQ ID NO: 1). Sequences from top to bottom are SEQ ID NOs: 36-41, 1, 7 and 42-44. (FIG. 12C) K514 15-mer epitopes analyzed in the IEDB MHC-I binding prediction tool showed HLA-C restricted binding that fell in the top 2% of binding affinities (RRRPVVGEF (SEQ ID NO: 9) and RRRPVVGEL (SEQ ID NO: 45), and similarly for (FIG. 12D) K624 products, the minimal epitope identified in the products (FPLLAECLF, SEQ ID NO: 1) was predicted to have a high affinity for HLA-B*35:01 based on the donor HLA (FPLLAECLL (SEQ ID NO: 46)).

FIGS. 13A-13B—Epitope specificities identified in (FIG. 13A) K929 and (FIG. 13B) K017 KST products. K929 KSTs were specific for v-IRF3 and v-Cyclin. K017 KSTs were specific for overlapping v-FLIP epitopes. CD4 and CD8 intracellular cytokine responses could not be determined for K929 and K017 KST products. Epitopes reported in graphs: (FIG. 13A) v-Cyclin: LDFWHHEVNTLITKA (SEQ ID NO: 6), v1RF3: LYHGNNPPKKFGVIC (SEQ ID NO: 5), vIRF3: DMVPLVIKLRLRSVT (SEQ ID NO: 15); and (FIG. 13B) IGSRSTPQTFLHWVY (SEQ ID NO: 28) and STPQTFLHWVYCMEN (SEQ ID NO: 29).

FIG. 14-HLA typing for each donor was conducted and reported here.

FIGS. 15A-15C—Characterization of Donor 031120 KSHV-specific T cells. (FIG. 15A) T cells were specific for KSHV vIL-6 in the CD45RA⁺ T cell fraction following three stimulations with dendritic cells and lacked specific responses in the (FIG. 15B) CD45 RA-negative fraction. PHA=positive control. (FIG. 15C) Epitope mapping of 031120 KSTs revealed specific responses for 2 overlapping vIL-6 epitopes: v-IL6 peptide 27: EVLFKFLTTEFGKSV (SEQ ID NO: 20) and 28: KFLTTEFGKSVINVD (SEQ ID NO: 21).

FIGS. 16A-16B—(FIG. 16A) Donor 031120 epitope specificities (EVLFKFLTTEFGKSV (SEQ ID NO: 20) and KFLTTEFGKSVINVD (SEQ ID NO: 21)) have sequences homologous to human IL6 (EVYLEYLQNRFESSE; SEQ ID NO: 47, default NIH BLAST search) reflected by sequence identity matching. (FIG. 16B) HLA typing of donor 031120 cells was used to (FIG. 16C) predict MHC-I binding using the IEDB prediction tool. These predicted epitopes (SEQ ID NOs: 48-49) were not confirmed in vitro.

FIGS. 17A-17B—In this iteration of KSHV-specific T cell generation, donor P001 CD45RA-negative T cells were expanded and demonstrated specificity for the vGPCR peptide pool. (FIG. 17A) T cells did not show specificity in the CD45RA⁺ fraction after two stimulations, however specificity was demonstrated in the (FIG. 17B) CD45RA-negative fraction post stimulation 3 (not shown) and post stimulation 4. To expand more P001 KST products post stimulation 3, a stimulation 4 was conducted with v-GPCR peptide-pulsed phytohemagglutinin (PHA) blasts as antigen-presenting cells. The resulting products showed robust IFNγ secretion for v-GPCR peptides (KQSLGWVLTSAALLI (SEQ ID NO: 22) and GWVLTSAALLIALVL (SEQ ID NO: 23) by ELISPOT assay.

FIGS. 18A-18C—Post stimulation 4, P001 KSTs were used in epitope mapping studies. (FIG. 18A) Overlapping v-GPCR 15-mer epitopes were identified in the products: KQSLGWVLTSAALLI (SEQ ID NO: 22) and GWVLTSAALLIALVL (SEQ ID NO: 23). Epitope specificities also share homology to human GPCR (HNYTIWSVLVAIWTV (SEQ ID NO: 50). Based on the (FIG. 18B) donor HLA, (FIG. 18C) 9-mer responses in the top 2% of binding affinities were identified using the IEDB MHC-I binding prediction tool (SLGWVLTSA (SEQ ID NO: 51); TSAALLIAL (SEQ ID NO: 52); SAALLIALV (SEQ ID NO: 53)).

FIG. 19-Cytolytic activity of P001 KSTs against autologous peptide-pulsed (actin=negative control) PHA blasts was conducted using chromium-51 release assay. After 24 hours, P001 KSTs showed greater killing of PHA blasts pulsed with v-GPCR peptides compared to actin.

FIG. 20-KST epitope specificities and predicted HLA associations. Minimal vFLIP epitopes identified in K624 products are as follows: TFPLLAECL (SEQ ID NO: 7) and FPLLAECLF (SEQ ID NO: 1). Minimal vIRF-3 epitopes identified in K514 products are as follows: RRRPVVGEF (SEQ ID NO: 9), RRPVVGEFL (SEQ ID NO: 10), PLVIKLRLR (SEQ ID NO: 11), LVIKLRLRS (SEQ ID NO: 12), and VIKLRLRSV (SEQ ID NO: 13). Specific vIRF-3 epitopes identified in K929 products are as follows: LYHGNNPPKKFGVIC (SEQ ID NO: 5) and DMVPLVIKLRLRSVT (SEQ ID NO: 15). Specific vCYCLIN epitopes identified in K929 products are as follows: LDFWHHEVNTLITKA (SEQ ID NO: 6). Specific vCYCLIN epitopes identified in K974 products are as follows: SKLRSLTPISTSSLC (SEQ ID NO: 4). Specific vIL-6 epitopes identified in 031120 products are as follows: EVLFKFLTTEFGKSV (SEQ ID NO: 20) and KFLTTEFGKSVINVD (SEQ ID NO: 21). Specific vGPCR epitopes identified in P001 products are as follows: KQSLGWVLTSAALLI (SEQ ID NO: 22) and GWVLTSAALLIALVL (SEQ ID NO: 23).

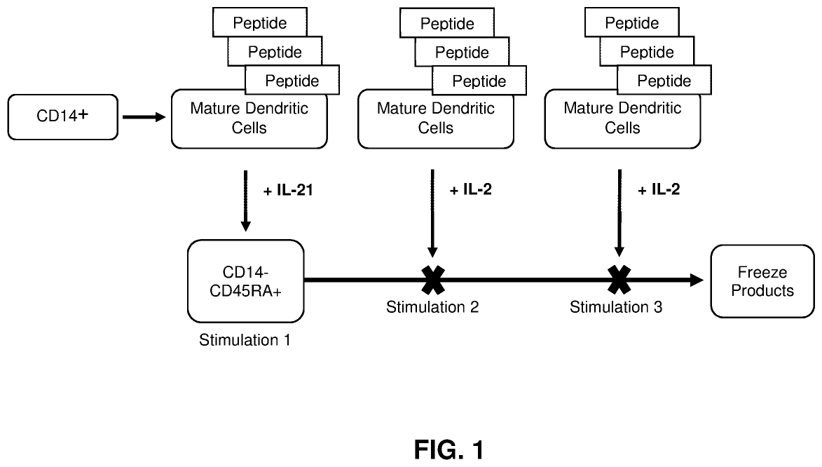

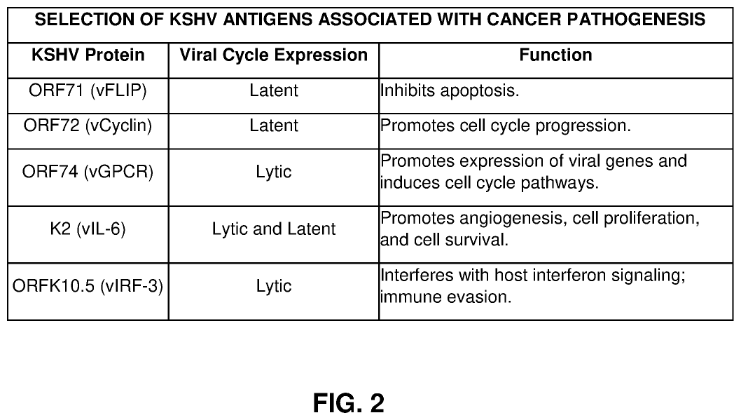

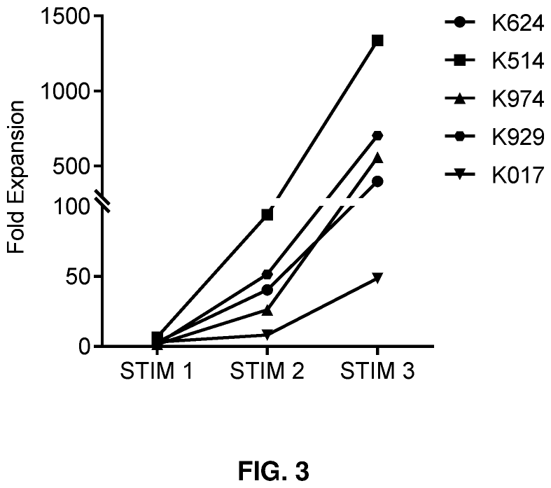

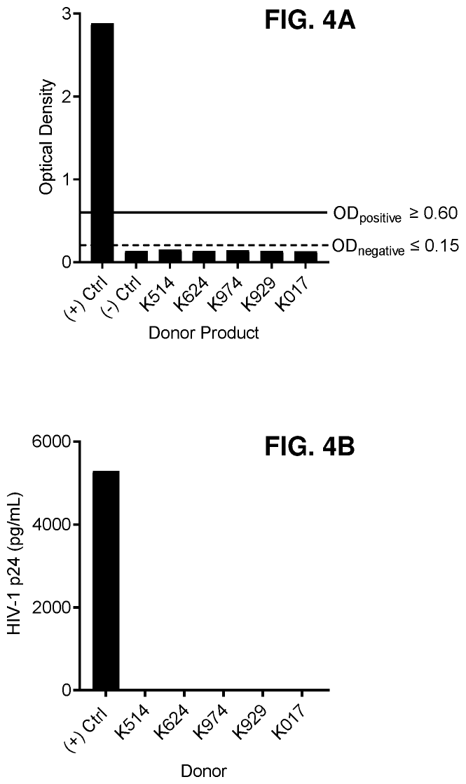

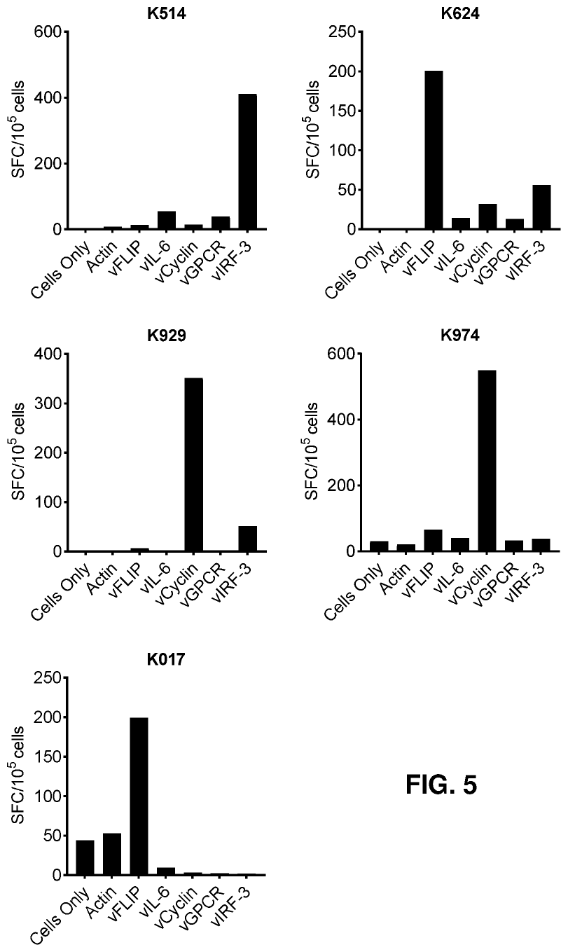

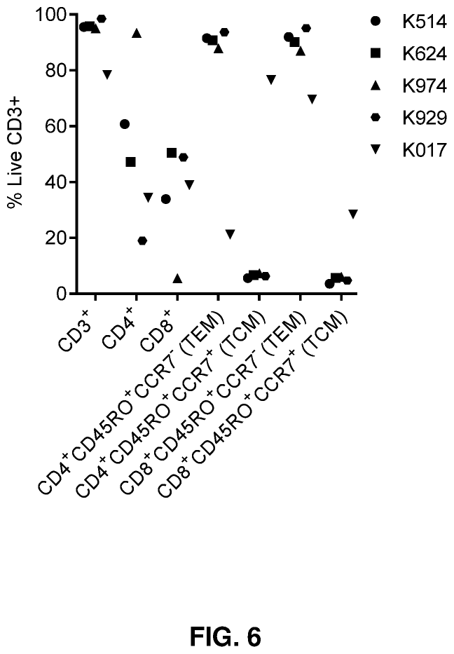

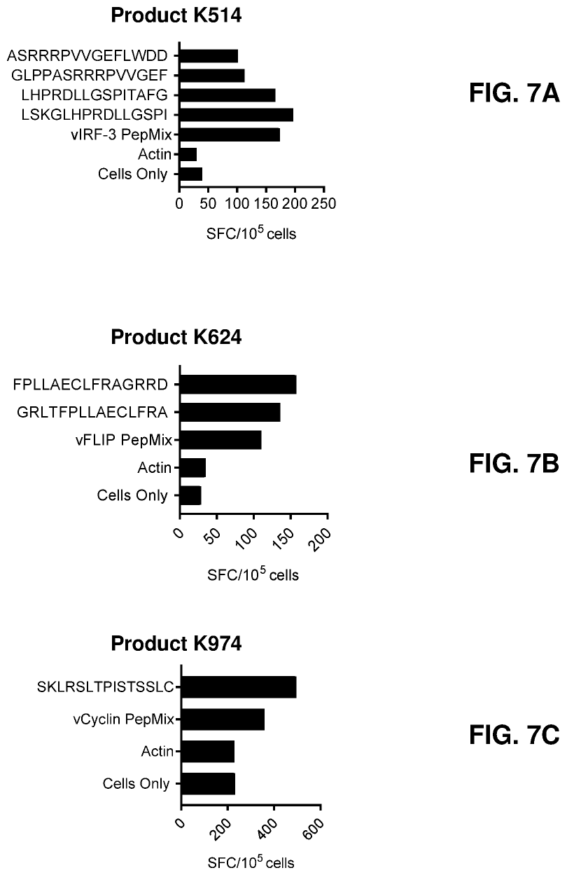

### DETAILED DESCRIPTION OF THE INVENTION

### 1. Overview

Applicants have successfully expanded KSHV-specific T cell (KST) products from KSHV and HIV naïve donors (n=5) against KSHV cancer associated antigens, and characterized epitope specific responses. It is believed that KSHV-specific T cell products expanded from virus-naïve donors eliciting these antigen-specific responses have not been previously described. The invention described herein provides evidence towards the development of allogeneic, or “off-the-shelf,” KSHV-specific T cell therapeutic as clinical efficacy of similar off-the-shelf virus-specific T cell products has been demonstrated. The epitopes elucidated may also contribute to ideas for epitope-based vaccine development for the prevention of KSHV infection and associated malignancy.

In particular, infusion of virus-specific T cells (VST) can enhance anti-viral immunity and augment immune responses to better protect against or treat viral infections and virus-mediated malignancies.

To develop an “off the shelf” VST product targeting KSHV antigens—naïve T cells, such as CD45RA-selected T cells—were isolated from peripheral blood mononuclear cells (PBMCs) derived from healthy donors, and expanded using dendritic cells pulsed with KSHV vFLIP, vIL-6, vCyclin, vGPCR, and vIRF-3 15-mer overlapping peptide pools (see generation schematic in the figures and examples). These antigens are oncoproteins that have shown to generate little to no immune responses in current published works. Moreover, a virus-specific T cell platform targeting these KSHV proteins has not been previously described and certainly not from healthy donors. Expansion of these cell products from the virus naïve cells has revealed novel epitopes that expands knowledge in the fields of cell therapy and vaccine design for infectious disease.

In other words, based on evaluation of KSHV-specific responses to critical antigens (e.g., v-FLIP, vIL-6 vCYCLIN, vGPCR, and vIRF-3) linked to the pathogenesis of KSHV associated cancers, Applicant has demonstrated herein that functional KSHV-specific T cells (KSTs) can be primed from virus naïve donors, and that their epitope repertoire will vary from T cell responses identified in seropositive individuals. The data presented herein further demonstrates that distinct epitope repertoires may be useful for the development of a novel allogeneic T cell therapeutic, or as vaccine targets.

Thus, in one apsect, the invention provides a pharmaceutical composition comprising a Kaposi sarcoma-associated herpesvirus (KSHV)-specific T cell (KST), wherein said KST: (1) derives from a naïve T cell that has not been previously exposed to KSHV antigens; (2) has been exposed to, presented with, and/or stimulated by a KSHV antigen or an epitope thereof; and (3) produces a cytokine produced by activated T lymphocytes, upon exposure to said KSHV antigen or said epitope thereof.

In certain embodiments, the KST is a human T cell.

In certain embodiments, the naïve T cell is isolated from PBMC of a subject naïve to KSHV (e.g., not previously infected by/exposed to KSHV), and optionally further naïve to HIV (e.g., not previously infected by/exposed to HIV).

In certain embodiments, the KSHV antigen comprises a KSHV cancer-associated antigen.

In certain embodiments, the KSHV antigen comprises vFLIP, vIL-6, vCyclin, vGPCR, vIRF-3, and/or a combination thereof.

In certain embodiments, the KST has been exposed to, presented with, and/or stimulated by said KSHV antigen or said epitope thereof through contacting a mature antigen-presenting cell (APC, e.g., a mature dendritic cell) that expresses/presents said KSHV antigen or said epitope thereof.

In certain embodiments, the KST is contacted by said APC in the presence of one or more cytokines conductive to antigen presentation and stimulation of T cells.

In certain embodiments, the one or more cytokines conductive to antigen presentation and stimulation of T cells comprise IL-2, IL-7, IL-15, IL-21.

In certain embodiments, the APC is a mature dendritic cell (i) isolated from PBMC as a CD14⁺ immature dendritic cell; (ii) pulsed with said KSHV antigen in the presence of one or more cytokines conductive for dendritic cell maturation (such as GM-CSF, IL-4, IL-6, IL-1β, TNFα, IFNγ, and prostaglandin E2 (PGE2)); and (iii) optionally irradiated (e.g., at 25 Gy).

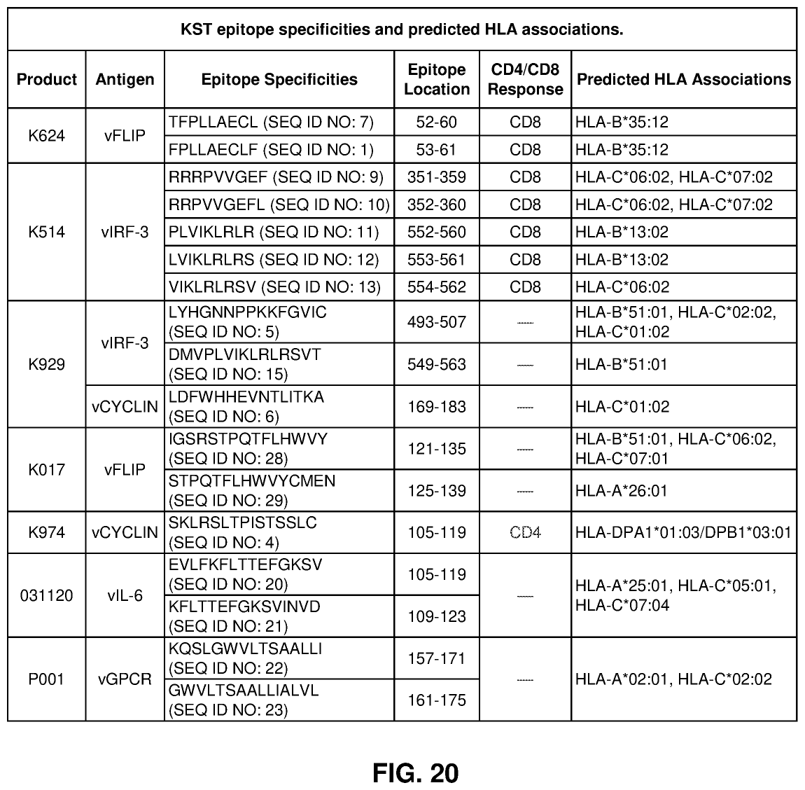

In certain embodiments, the epitope thereof comprises any one or more of the epitopes in FIG. 20, and any one more more of SEQ ID NOs: 1-6 (e.g., any one or more of SEQ ID NOs: 1-29).

In certain embodiments, the cytokine produced by activated T lymphocytes comprises IFNγ and/or TNFα.

In certain embodiments, the naïve T is a CD14⁻CD45RA⁺ naïve T.

Another aspect of the invention provides a method of treating a disease caused by or associated with Kaposi sarcoma-associated herpesvirus (KSHV), in a subject in need thereof, the method comprising administering an effective amount of the pharmaceutical composition of claim 1 to the subject, such that the disease is treated.

In certain embodiments, the disease is Kaposi sarcoma (KS), primary effusion lymphoma (PEL), and/or multicentric Castleman's disease (MCD).

In certain embodiments, the subject a human.

In certain embodiments, the human is immune compromised.

In certain embodiments, the human is HIV-positive, such as a human receiving highly active antiretroviral therapy (HAART) for the treatment of HIV who exhibits CD4 immune reconstitution.

In certain embodiments, the the human is a transplant recipient, and/or is under treatment by an immunosuppressant (such as one or more glucocorticoids, cytostatics, antibodies, and/or drugs acting on immunophilins).

In certain embodiments, the the subject is co-infected by KSHV and an additional virus (such as HIV, EBV, HHV6, and/or CMV).

In certain embodiments, the subject is HLA-matched with the HLA-type of the KST in said pharmaceutical composition.

Another aspect of the invention relates to a method of producing a Kaposi sarcoma-associated herpesvirus (KSHV)-specific T cell (KST), the method comprising: contacting a naïve T cell that has not been previously exposed to KSHV antigens with a KSHV antigen or an epitope thereof.

In certain embodiments, the the KST is a human T cell.

In certain embodiments, the the naïve T cell is isolated from PBMC of a subject naïve to KSHV (e.g., not previously infected by/exposed to KSHV), and optionally further naïve to HIV (e.g., not previously infected by/exposed to HIV).

In certain embodiments, the KSHV antigen comprises a KSHV cancer-associated antigen.

In certain embodiments, the KSHV antigen comprises vFLIP, vIL-6, vCyclin, vGPCR, vIRF-3, and/or a combination thereof.

In certain embodiments, the naïve T cell is contacted by said KSHV antigen or said epitope thereof through contacting a mature antigen-presenting cell (APC, e.g., a mature dendritic cell) that expresses/presents said KSHV antigen or said epitope thereof.

In certain embodiments, the naïve T cell is contacted by said APC in the presence of one or more cytokines conductive to antigen presentation and stimulation of T cells.

In certain embodiments, the one or more cytokines conductive to antigen presentation and stimulation of T cells comprise IL-2, IL-7, IL-15, IL-21.

In certain embodiments, the APC is a mature dendritic cell (i) isolated from PBMC as a CD14⁺ immature dendritic cell; (ii) pulsed with said KSHV antigen in the presence of one or more cytokines conductive for dendritic cell maturation (such as GM-CSF, IL-4, IL-6, IL-1β, TNFα, IFNγ, and prostaglandin E2 (PGE2)); and (iii) optionally irradiated (e.g., at 25 Gy).

In certain embodiments, the epitope thereof comprises any one or more of the epitopes in FIG. 20, and any one more more of SEQ ID NOs: 1-6 (e.g., any one or more of SEQ ID NOs: 1-29).

In certain embodiments, the method further comprises verifying production of a cytokine produced by activated T lymphocytes (such as IFNγ and/or TNFα) upon exposing said KST to said KSHV antigen or said epitope thereof.

In certain embodiments, the mature dendritic cell has an HLA type that matches said KSHV antigen or said epitope thereof based on FIG. 20.

In certain embodiments, the naïve T cell is a CD14⁻CD45RA⁺ naïve T.

### 2. Definitions

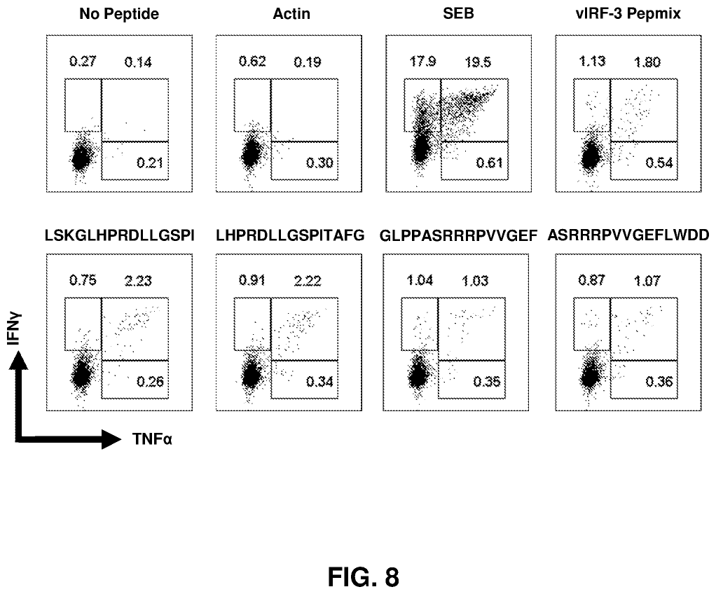

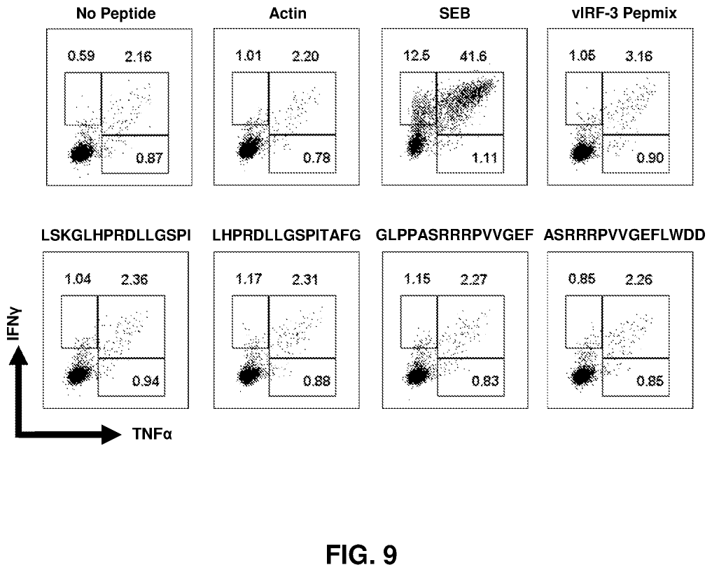

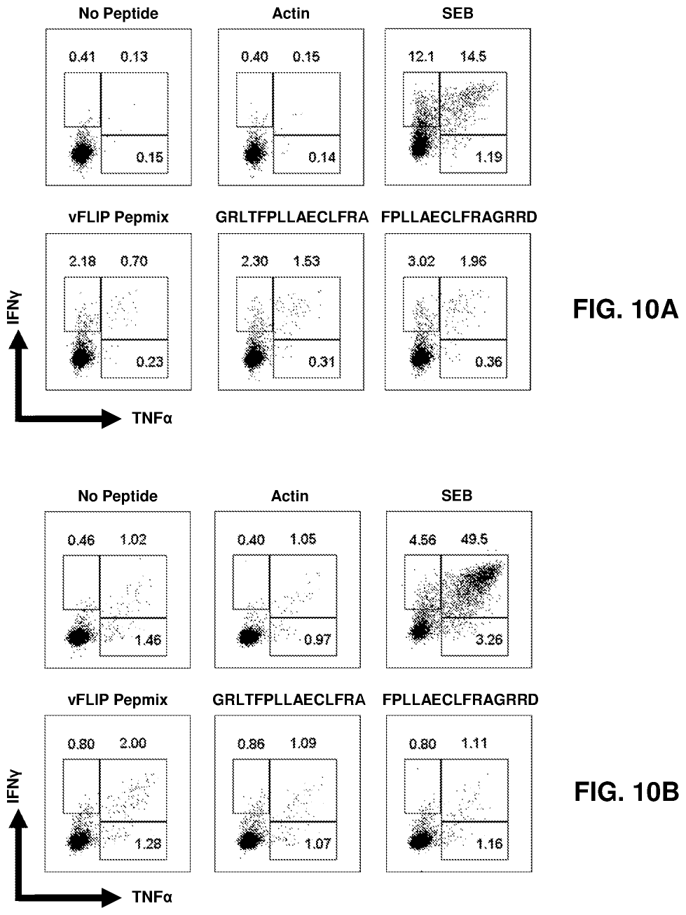

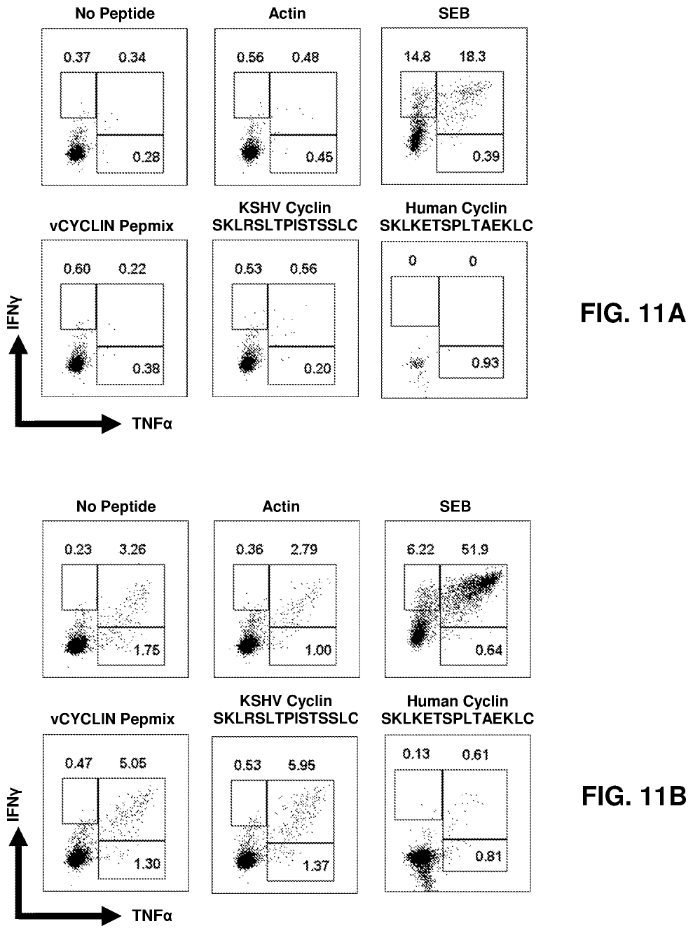

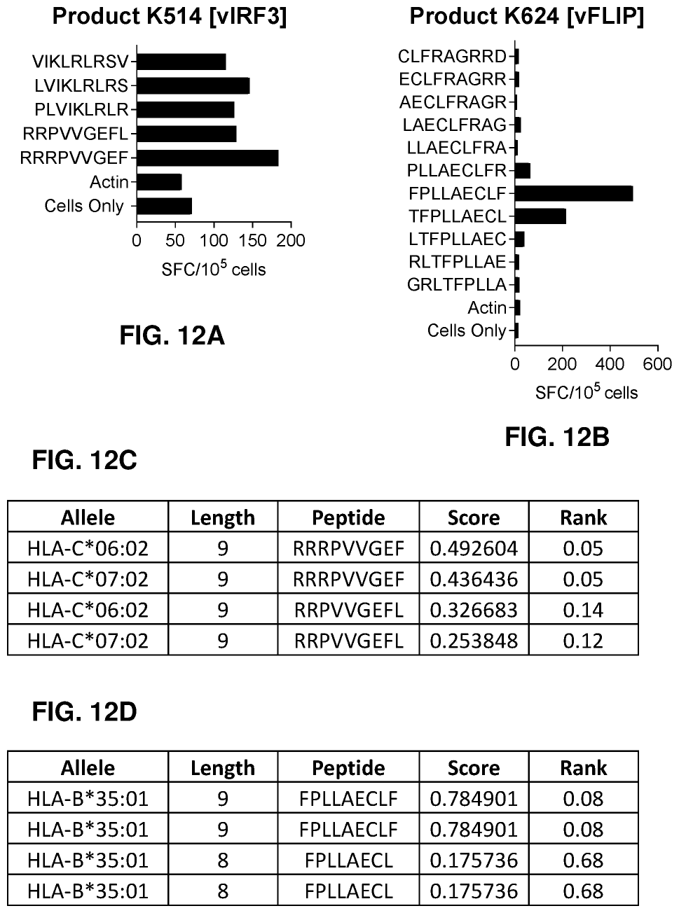

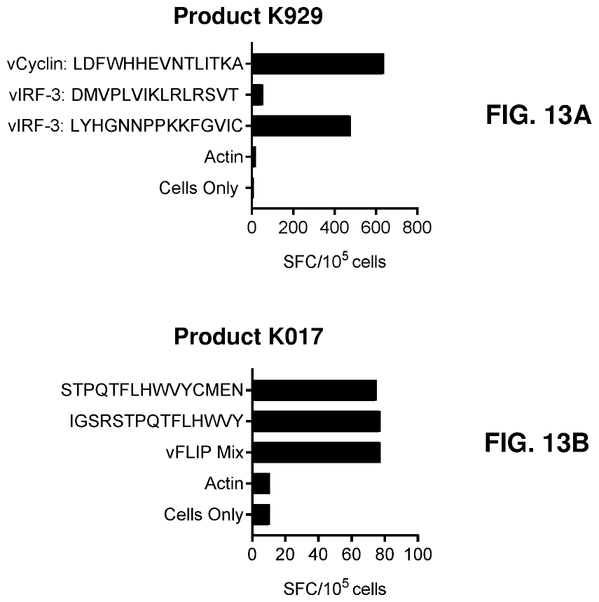

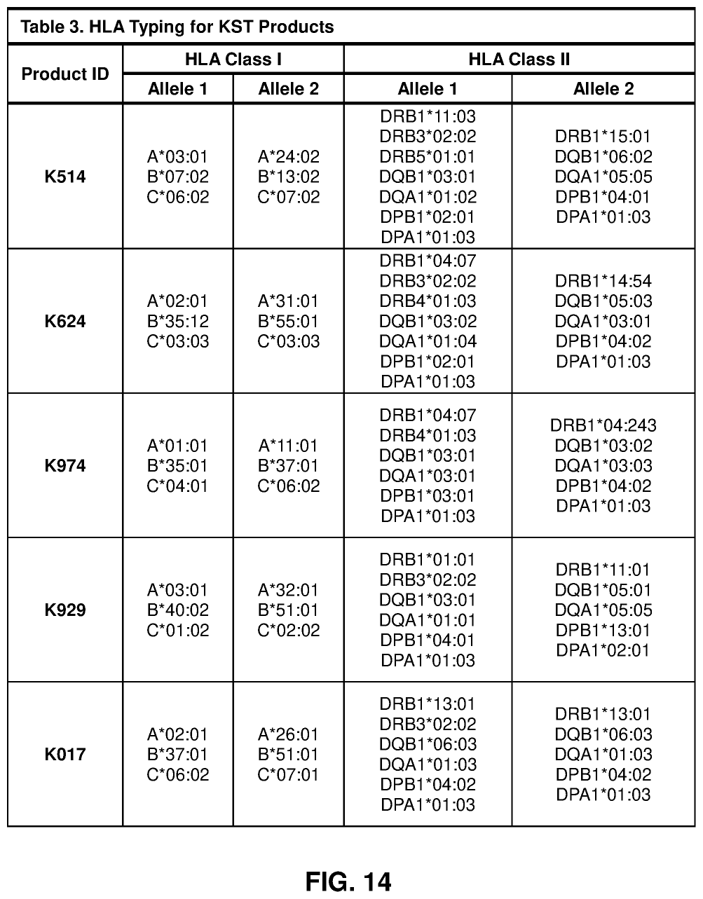

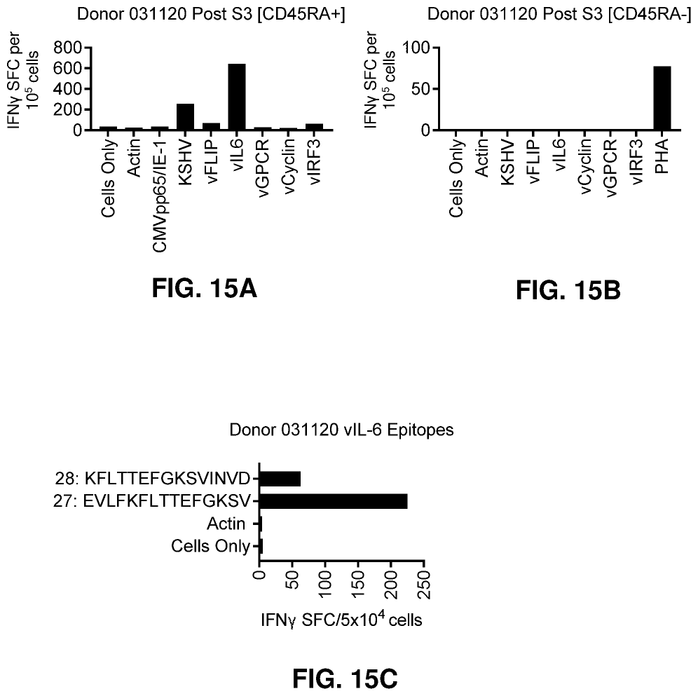

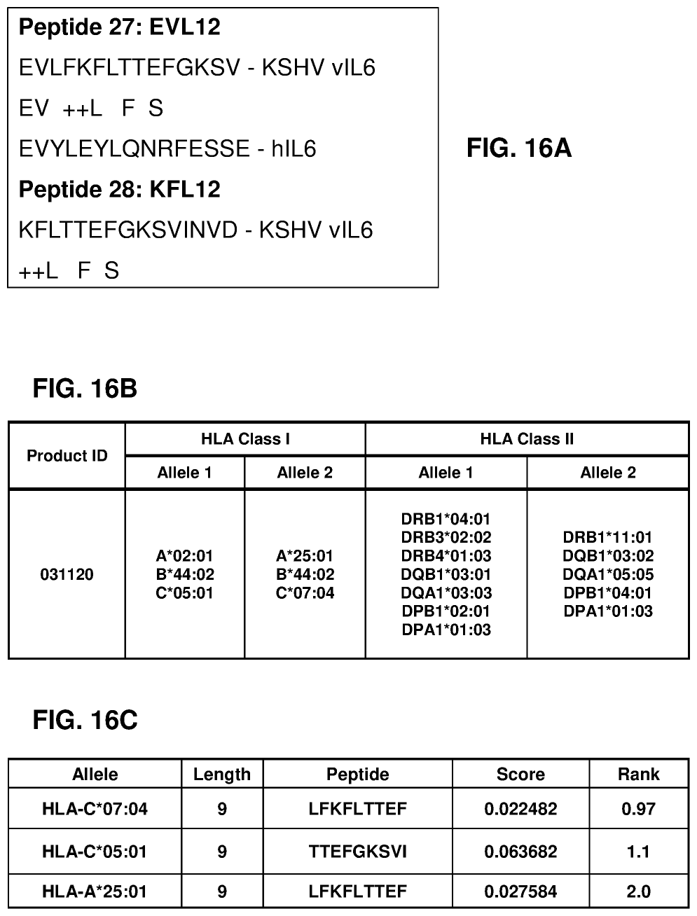

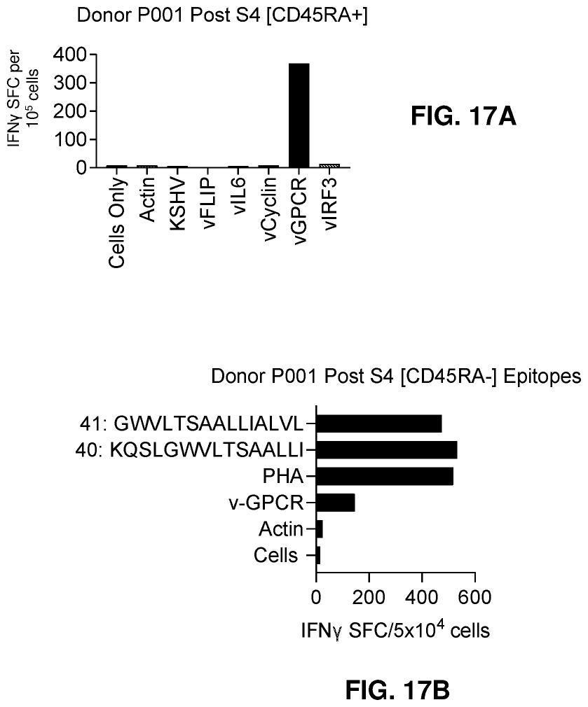

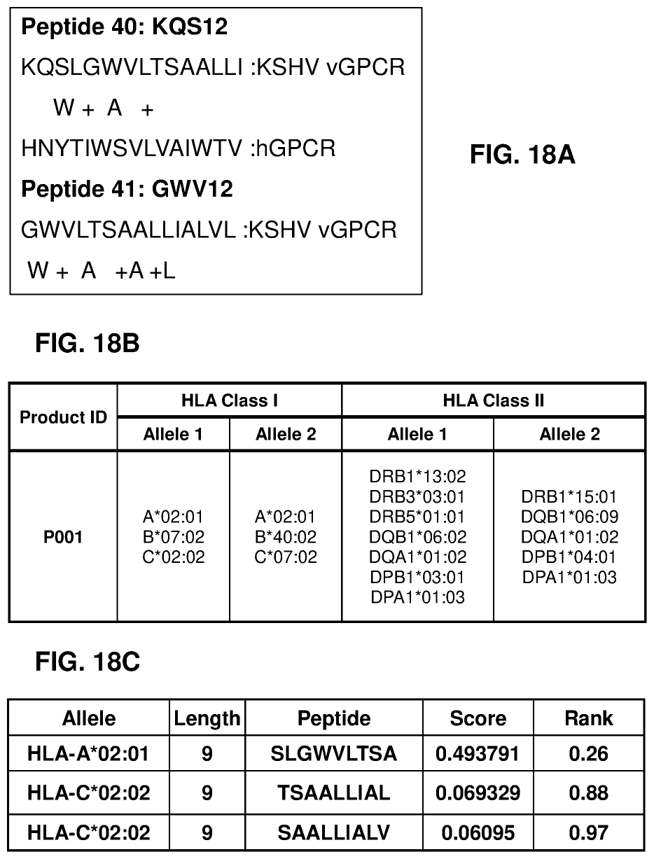

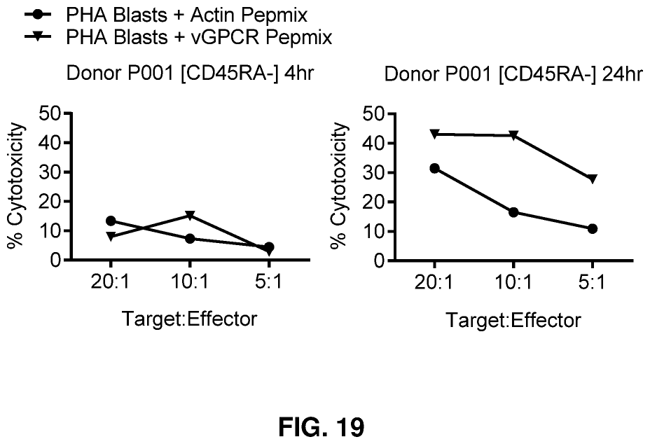

T cells. A T cell is a type of lymphocyte, which develops in the thymus gland and plays a central role in the immune response. T cells can be distinguished from other lymphocytes by the presence of a T-cell receptor on the cell surface. These immune cells originate as precursor cells, derived from bone marrow, and develop into several distinct types of T cells once they have migrated to the thymus gland. T cell differentiation continues even after they have left the thymus. FIG. 8 describes subtypes of T cells.

Groups of specific, differentiated T cells have an important role in controlling and shaping the immune response by providing a variety of immune-related functions.

One of these functions is immune-mediated cell death, and it is carried out by T cells in several ways: CD8⁺ T cells, also known as “killer cells”, are cytotoxic this means that they are able to directly kill virus-infected cells as well as cancer cells. CD8⁺ T cells are also able to utilize small signaling proteins, known as cytokines, to recruit other cells when mounting an immune response.

A different population of T cells, the CD4⁺ T cells, function as “helper cells”. Unlike CD8⁺ killer T cells, these CD4⁺ helper T cells function by indirectly killing cells identified as foreign: they determine if and how other parts of the immune system respond to a specific, perceived threat. Helper T cells also use cytokine signaling to influence regulatory B cells directly, and other cell populations indirectly.

Regulatory T cells are yet another distinct population of these cells that provide the critical mechanism of tolerance, whereby immune cells are able to distinguish invading cells from “self” thus preventing immune cells from inappropriately mounting a response against oneself (which would by definition be an “autoimmune” response). For this reason these regulatory T cells have also been called “suppressor” T cells. These same self-tolerant cells are co-opted by cancer cells to prevent the recognition of, and an immune response against, tumor cells.

Subpopulations of T cells which may be separated or enriched and used in the methods disclosed herein.

Naïve T cell. This term describes T cells which have not yet encountered their specific antigen. In peripheral lymphoid organs naïve T lymphocytes can interact with antigen presenting cells (APCs), which use an MHC molecule to present antigen. If the T lymphocyte recognizes a specific antigen, it will proliferate and differentiate into effector T lymphocytes of a particular type. In contrast, a subject who is naïve to KSHV-antigens includes one who has not been infected by KSHV, who has not developed a cancer expressing KSHV-antigens, and whose immune system is tolerant to or does not substantially recognize KSHV-antigens.

Precursor T cell. This term describes cells which can differentiate or be induced to differentiate into T cells. It includes multipotential hematopoietic stem cells (hemocytoblasts), common lymphoid progenitors, and small lymphocytes.

The term “isolated” means separated from components in which a material is ordinarily associated, for example, an isolated PBMC population can be separated from red blood cells, plasma, and other components of blood and an isolated T cell can be separated or substantially separated from other types of leukocytes.

A “control” is a reference sample of subject used for purposes of comparison with a test sample or test subject. Positive controls measure an expected response and negative controls provided reference points for samples where no response is expected.

“Cord blood” has its normal meaning in the art and refers to blood that remains in the placenta and umbilical cord after birth and contains hematopoietic stem cell. Cord blood may be fresh, cryopreserved, or obtained from a cord blood bank. It is one source of cells that can be HLA-matched to a subject or patient for production of KSHV-specific T cells. Thus, in some embodiments, the naïve T cells can be isolated form cord blood.

Peptide epitopes of Kaposi sarcoma-associated herpesvirus (KSHV) antigens. These include peptides having unmodified (natural) amino acid sequences which correspond to fragments of a longer KSHV antigen's amino acid sequence that when presented by an HLA class 1 or HLA class 2 molecules are recognized by T cells. Additionally, in some embodiments, this term encompasses modified peptides, such as peptides with modified C or N terminal residues, modified amino acid side-chains, or other modified peptides disclosed herein, which are HLA restrictable and which are recognized by T cells which also recognize a corresponding unmodified amino acid sequence. Peptide epitopes of KSHV antigens may also be present on longer peptides, such as those comprising the amino acid sequences of any one of SEQ ID NOs: 1-29 (such as any one of SEQ ID NOs: 1-6 or 1-23), e.g., peptides having a length up to 15, 20, 25, 30, 35, 40, 45, 50 or more amino acid residues and which can be processed and restricted by, or directly restricted by or bound to, HLA class 1 or HLA class 2 molecules. In some embodiments, a KSHV antigen HLA class 1 restricted peptide epitope will consist of 8, 9 or 10 contiguous residues of the KSHV antigen and a KSHV antigen HLA class 2 restricted peptide epitope will consist of 13, 14, 15, 16, 17 or 18 contiguous residues of the KSHV antigen. Longer peptides may be internalized by an antigen presenting cell and processed into shorter peptides comprising a KSHV antigen epitope that can complex with HLA class 1 or class 2 molecules. Peptide epitopes also include truncated versions of the peptides consisting of the amino acid sequences of SEQ ID NOs: 1-29 (such as any one of SEQ ID NOs: 1-6 or 1-23) which retain a capacity to be HLA class 1 or HLA class 2 restricted and recognized by T cells.

HLA haplotypes. Common HLA haplotypes are described by, and incorporated by reference to, Pedron, B., et al. Common genomic HLA haplotypes contributing to successful donor search in unrelated hematopoietic transplantation. BONE MARROW TRANSPLANT 31, 423-427 (2003). https://doi.org/10.1038/sj.bmt.1703876; Maiers, M., et al., High resolution HLA alleles and haplotypes in the US population. HUMAN IMMUNOLOGY, 2007, 68, 779-788; and to Hurley, C. K. et al., Common, intermediate and well-documented HLA alleles in world populations: CIWD version 3.0.0., HLA IMMUN. RESP. GEN. 2020, 95(60: 503-637. Some common HLA haplotypes include HLA-A1, HLA-A2, HLA-A3, HLA-A24, HLA-A68, HLA-B7, HLA-B8, HLA-B35, HLA-B44, HLA-B60, HLA-B61 and HLA-B62.

The HLA alleles described herein are expressed in codominant fashion. This means the alleles (variants) inherited from both parents are expressed equally. Each person carries 2 alleles of each of the 3 class-I genes, (HLA-A, HLA-B and HLA-C), and so can express six different types of HLA class I molecules or antigens.

In the HLA class II locus, each person inherits a pair of HLA-DP genes (DPA1 and DPB1, which encode α and β chains), a couple of genes HLA-DQ (DQA1 and DQB1, for α and β chains), one gene HLA-DRα (DRA1), and one or more genes HLA-DRβ (DRB1 and DRB3, -4 or -5). That means that one heterozygous individual can inherit six or eight functioning class II alleles, three or more from each parent. The role of DQA2 or DQB2 is not verified. The DRB2, DRB6, DRB7, DRB8 and DRB9 are pseudogenes.

The KSHV antigen peptides disclosed herein will bind to one or more of the HLA class 1 or class 2 MHC molecules described herein. There are two types of HLA molecules, class 1 and class 2, and both are highly polymorphic. The core binding subsequence of both HLA class 1 and 2 is approximately 9 amino acids long. However, HLA class 1 molecules rarely bind peptides much longer than 9 amino acids, while HLA class 2 molecules can accommodate longer peptides of 10-30 residues. One skilled in the immunological arts may select a peptide antigen length suitable for binding to HLA class 1 or class 2 molecules.

In some embodiments, the HLA class 1 or class 2 restricted KSHV antigen peptides disclosed herein, or shorter fragments thereof, may range in length from 6, 7, 8, 9, 10, 11, 12, 13, 14, 15, 16, 17, 18, 19, 20, 21, 22, 23, 24, 25, 26, 27, 28, 29 or 30 amino acid residues. In some embodiments, a peptide epitope, such as those comprising SEQ ID NOs: 1-29 (such as any one of SEQ ID NOs: 1-6 or 1-23), may be processed by an antigen presenting cells prior to its association with an HLA class 1 or class 2 molecule. Such processing may decrease its peptide length. Antigen processing and presenting machinery and mechanisms processing MHC class 1 presented peptides (such as those presented by HLA class 1 molecules), is known in the art and incorporated by reference to Leoni, P. et al., MHC Class I Antigen Processing and Presenting Machinery: Organization, Function, and Defects in Tumor Cells, J. Nat. Cancer Inst. 105(16): 1172-1187; and to Roche, P. A., et al., The ins and outs of MHC class II-mediated antigen processing and presentation, NATURE REVIEWS, 2015, 15, 203-216. Antigen-presenting cells (APC), including B cells and dendritic cells, present the peptides to cytotoxic T cells. Peptides (regardless of length) can be presented by virtually any cell type as they require minimal processing by endonucleases to produce the 8-10mer peptides required to be presented by HLA class I molecules expressed by the APC.

HLA Matching. A subject may be matched to a donor at least one HLA haplotype (e.g., HLA-A1), HLA allele or synonymous allele (e.g., HLA-A*02, HLA-B*07), or by common expression of at least one specific HLA protein (e.g., HLA-A*02:101, HLA-B*07:01). Preferred matching occurs at the level of an allele group.

Matching may be performed by methods known in the art which include genetic or serological procedures to determine whether the donor and subject share an HLA allele, allele group, or specific HLA protein. PCR (polymerase chain reaction) and NGS (next generation sequencing) HLA typing methods are known and commercially available for HLA genotyping the HLA class I and class II gene polymorphisms for an individual, for example, from CD Genomics (cd-genomics dot com slash Genotyping dot html) and others.

In some embodiments, a donor may be a close family member, such as a parent, sibling, son or daughter, uncle or aunt, grandparent, cousin, who shares with a recipient, an appropriate HLA class 1 or class 2 molecule that restricts a peptide epitope of KSHV antigen. In some other embodiments, a donor may be unrelated by blood (e.g., not a relative or family member).

HLA class 1 and HLA class 2 antigen processing and presentation. HLA (human leukocyte antigens) are major histocompatibility (MHC) molecules.

HLA class 1 molecules comprise a polymorphic alpha chain and beta-2 microglobulin which forms a complex when a peptide, such as a peptide epitope of SEQ ID NOS: 1-26, binds to the alpha chain. All nucleated cells express HLA class 1 molecules. Cytotoxic (CD8⁺) T cells are able to respond to an HLA class I peptide complex. In nature, peptides presented by HLA class 1 molecules are typically generated by the cytosolic proteasome and loaded on a class 1 molecule in the endoplasmic reticulum. Usually, HLA class 1 molecules or complexes bind to peptide antigens ranging in length from 8-10 amino acid residues. In some embodiments, antigen presenting cells may be pulsed with peptides that directly bind to HLA class 1 molecules and form a HLA-peptide complex or, alternatively, are internalized, processed and presented as part of an HLA-peptide complex.

HLA class 2 molecules comprise polymorphic alpha and beta chains which together bind a peptide and form a complex recognizable by T helper (CD4⁺) cells. Dendritic cells, mononuclear phagocytes, some endothelial cells, and thymic epithelium express HLA class 2 molecules. In nature, peptides presented by HLA class 2 molecules are usually derived from proteins present in endosomes or lysosomes which often are internalized from the extracellular medium. Cellular proteases such as cathepsin generate peptides from these proteins which are presented by the HLA class 2 complex. Usually, HLA class 2 molecules or complexes bind to peptide antigens ranging in length from 13-18 amino acid residues. In some embodiments, antigen presenting cells may be pulsed with peptides that directly bind to HLA class 2 molecules and form a HLA-peptide complex or, alternatively, are internalized, processed and presented as part of an HLA-peptide complex.

Methods for producing T cells ex vivo that recognize antigens such as KSHV antigens are incorporated by reference to using the steps disclosed by U.S. Pat. No. 9,885,021, by U.S. Pat. No. 10,934,525, or by PCT/US2016/023413 which are incorporated by reference for all purposes. These T cells may be produced using autologous donor cells (e.g., from a patient's own bone marrow or cord blood) or using cells from a donor who shares 1, 2, 3, 4, 5, 6 or more MHC class I or class II molecules such as the HLA molecules mentioned above. T cells produced ex vivo to a KSHV antigen peptide may be administered to a fully histocompatible (e.g., autologous, or identical twin) or partially histocompatible (e.g., someone who shares at least one HLA class 1 or class 2 allele or protein, but not all HLA alleles or proteins, with a donor).

Off the shelf T cells. These are ready-to-use, off-the-shelf therapeutic T cells. These can be produced using the KSHV antigen epitopes described herein, usually from the blood cells from normal, healthy donors who are at least partially HLA matched to a subject undergoing treatment. Such off-the-shelf T cells are typically well characterized as to origin and HLA background and by an ability to kill KSHV-associated cancer cells expressing KSHV antigens. In many embodiments, the T cells are cryo-preserved—stored frozen in liquid nitrogen—until it's time to use them. In one embodiment, a cancer patient visits a physician where the cancer is identified to be KSHV-associated and where the subject's HLA background is determined or referenced. With the identity of the cancer-specific or cancer-associated antigens and the subject's HLA background in hand, the physician visits a T cell bank filled with large below zero freezers and selects a banked T cell sample suitable for therapeutic use against the particular cancer in a subject having a particular HLA background. These “off-the-shelf,” ready-made cells are thawed, prepared or expanded and infused into the patient several days later to recognize and destroy the patient's KSHV-associated cancer cells expressing KSHV antigens.

### 3. Compositions and Methods of Producing KST

In certain aspects, the present disclosure provides methods for producing a Kaposi sarcoma-associated herpesvirus (KSHV)-specific T cell (KST), comprising contacting a naïve T cell or a T cell precursor that has not been exposed to KSHV antigens with a KSHV antigen or an epitope thereof.

In some embodiments, said naïve T cell or T cell precursor is contacted with said KSHV antigen or said epitope thereof through contacting a mature antigen presenting cell (e.g., a mature dendritic cell) that expresses or presents said KSHV antigen or epitope thereof.

In some embodiments of this method, the naïve T cell or precursor T cell is contacted with an antigen presenting cell and an KSHV antigen or epitope thereof using the steps disclosed by U.S. Pat. No. 9,885,021, by U.S. Pat. No. 10,934,525, or by PCT/US2016/023413, incorporated herein by reference, whose steps may be modified to replace an overlapping peptide library with a less complex mixture of one, two, three or more KSHV antigens or epitopes thereof, such as those described by SEQ ID NOs: 1-29 (such as any one of SEQ ID NOs: 1-6 or 1-23).

In some preferred embodiments, the antigen presenting cell is a dendritic cell. In some embodiments, the antigen present cell is derived from a CD14⁺ immature dendritic cell isolated from peripheral blood mononuclear cells.

In some embodiments, the antigen presenting cell, e.g., dendritic cell, is pulsed with at least one KSHV antigen or epitope thereof, such as one comprising any of the peptide of SEQ ID NOs: 1-29 (such as any one of SEQ ID NOs: 1-6 or 1-23). Methods for pulsing dendritic cells with an antigen is known in the art and can be found in, e.g., WO2016154112A, Celluzi et al. (“Peptide-pulse dendritic cells induce antigen-specific CT:-mediated protective tumor immunity”, J. Exp. Med. 1996, 183:283-287), and Gu et al. (“Ex vivo pulsed dendritic cell vaccination against cancer”, Acta Pharmacologica Sinica 2020, 41:959-969), incorporated herein by reference.

In some embodiments, the antigen presenting cell, e.g., dendritic cell, is pulsed with at least one KSHV antigen or epitope thereof in the presence of one or more cytokines conductive for dendritic cell maturation (such as GM-CSF, IL-4, IL-6, IL-1β, TNFα, IFNγ, and prostaglandin E2 (PGE2)).

In some embodiments, the antigen presenting cell is irradiated (e.g., at 25 Gy).

In some embodiments, a plurality of naïve T cells or T cell precursors are contacted with a plurality of antigen presenting cells that express or present the same KSHV antigen or epitope thereof. In some embodiments, a plurality of naïve T cells or T cell precursors are contacted with a plurality of antigen presenting cells pulsed with the same KSHV antigen or epitope thereof.

In other embodiments, a plurality of naïve T cells or T cell precursors are contacted with a plurality of antigen presenting cells that express or present the more than one KSHV antigens or epitopes thereof. In some embodiments, a plurality of naïve T cells or T cell precursors are contacted with a plurality of antigen presenting cells pulsed with more than one (e.g., at least 2, at least 3, at least 4, at least 5, at least 6) KSHV antigen or epitope thereof.

In some embodiments, the naïve T cell or T cell precursor is contacted with the antigen presenting cell in the presence of one or more cytokines conductive to antigen presentation and stimulation of T cells. In some embodiments, the one or more cytokines conductive to antigen presentation and stimulation of T cells are selected from the group consisting of IL-2, IL-7, IL-15, IL-21, and any combination thereof.

In some embodiments, the naïve T cell or T cell precursor is contacted with the antigen presenting cell more than once. In some embodiments, the naïve T cell or T cell precursor is contacted with the antigen presenting cell at least 2 times, at least 3 times, at least 4 times, or at least 5 times.

In some embodiments, the naïve T cell or T cell precursor is contacted with the antigen presenting cell (e.g., dendritic cell) presenting the KSHV antigen or epitope thereof at a ratio of about 1:1, about 2:1, about 3:1, about 4:1, about 5:1, about 6:1, about 7:1, about 8:1, about 9:1, or about 10:1.

In some embodiments, the KSHV antigen comprises a KSHV cancer-associated antigen.

In some embodiments, the KSHV antigen comprises vFLIP, vIL-6, vCyclin, vGPCR, vIRF-3, or a combination thereof.

In some embodiments, unmodified peptides comprising, consisting essentially of, or consisting of the amino acid sequences disclosed herein (e.g., SEQ ID NOs: 1-29 (such as any one of SEQ ID NOs: 1-6 or 1-23)) are used in this method as KSHV antigen or epitope thereof. In other embodiments, modified peptides, such as peptides having 1, 2 or 3 deletions, insertions or substitutions of amino acid residues in SEQ ID NOs: 1-29 (such as any one of SEQ ID NOs: 1-6 or 1-23) or other peptides disclosed herein; or covalently modified peptides may be used as KSHV antigen or epitope thereof in this method.

In some embodiments, the naïve T cell is isolated from peripheral blood mononuclear cells (PMBCs) of a donor or subject naïve to KSHV (e.g., not previously infected by or exposed to KSHV). In some embodiments, the donor or subject is further naïve to human-immunodeficiency virus (HIV) (e.g., not previously infected by or exposed to HIV).

In some embodiments, the naïve T cell or precursor T cell is a human cell.

In some embodiments, the naïve T cell or precursor T cell is autologous. In other embodiments, the naïve T cell or precursor T cell is HLA-type matched with the HLA-type of the intended recipient. In some embodiments, the naïve T cell or precursor T cell shares at least on, two, three, four, five, six, seven, eight or more HLA class 1 or HLA class 2 antigens with the intended recipient.

In some embodiments, the naïve T cell is a CD14⁻CD45RA⁺ naïve cell.

In some embodiments, the methods further comprise confirming specificity and cytotoxicity of the KST against the KHSV antigen or epitope thereof by verifying production of a cytokine produced by activated T-lymphocytes (such as IFNγ and or TNFα) upon exposing said KST to said KSHV antigen or said epitope thereof.

This method may further comprise separating the T cells which recognize a KSHV antigen or epitope thereof into subpopulations of T cells expressing one or more markers distinctive for that subpopulation, e.g., CD4+ and CD8+ T cell. Separation may be performed using methods known in the art including cell sorting, flow cytometry, or isolation of subpopulations based on different density or differential expression of T cell markers. Preferably, adoptive transfer of a polyclonal population of CD4⁺ and CD8⁺ KSHV-specific T cells is used in a third-party off-the-shelf setting, in which the T cells have epitope-specific activity through shared HLA allele(s) with the recipient, to support the in vivo persistence and expansion of transferred T cells. The ability to select KSHV-specific T cell products sharing HLA allele(s) with a patient, in a third party, off-the-shelf setting provides a clinical advantage over prior methods that do not provide ready access to these T cell products.

Some embodiments of this method further comprise suspending the KSTs cells in a storage buffer or in a cryogenic medium, and storing or freezing and thawing viable T cells for later use. Cryogenic media for freezing and thawing T cells are known and commercially available for example from Thermofisher and methods for freezing and recovering viable T cells are known. Culture media and media components for growing T cells are also known and commercially available, for example, from Thermofisher (www dot thermofisher dot com slash us slash en slash home slash life-science slash cell-culture slash mammalian-cell-culture slash specialty-media slash t-cell-media dot html (last accessed May 11, 2021, incorporated by reference) or Cell Culture Dish (cellculturedish dot com slash t-cell-media-comprehensive-guide-key-components (last accessed May 11, 2021, incorporated by reference).

In another aspect, the present disclosure provides a pharmaceutical composition comprising a Kaposi sarcoma associated herpesvirus (KSHV)-specific T cell (KST) produced by the methods described herein.

In some embodiments, the pharmaceutical composition further comprises an adjuvant or a cytokine.

Another aspect of this present disclosure is directed to a peptide or covalently modified peptide comprising or incorporating an amino acid sequence of a KSHV antigen or epitope thereof, such as an amino acid sequence comprising any one of SEQ ID NOs: 1-29 (such as any one of SEQ ID NOs: 1-6 or 1-23) or other peptides disclosed herein.

A peptide comprising and one of SEQ ID NOs: 1-29 (such as any one of SEQ ID NOs: 1-6 or 1-23) (such as any one of SEQ ID NOs: 1-6 or 1-23) may be covalently modified or engineered to improve its pharmacokinetic or pharmacodynamics properties, such as to increase its half-life in vivo or in vitro or resistance to excretion or degradation or its interaction with HLA molecules and T cell receptors.

A modification may be a covalent modification of the peptide's N- or C-terminal, covalent conjugation to PEG, an adjuvant, or another carrier, or the incorporation of one or more D-amino acid residues into the sequence.

A peptide complex comprising a peptide, such as those of SEQ ID NOs: 1-29 (such as any one of SEQ ID NOs: 1-6 or 1-23), may be formed by non-covalently binding a peptide to another moiety such as a carrier, adjuvant or substrate. In some embodiments a peptide is altered by non-covalently binding it to a carrier, adjuvant or substrate such as to PEG, BSA, or KLH. A peptide of SEQ ID NOs: 1-29 (such as any one of SEQ ID NOs: 1-6 or 1-23) may form a non-covalent complex with an MHC class I or class II molecule or a complex with a cell membrane or cell comprising MHC class 1 or 2 molecules.

A modification may also involve deleting, substituting or inserting at least 1, 2 or 3 amino acids into an amino acid sequence consisting of SEQ ID NOs: 1-29 (such as any one of SEQ ID NOs: 1-6 or 1-23) or the other amino acid sequences disclosed herein.

Another aspect of the disclosed technology is directed to use of a peptide or covalently modified peptide as described herein for the manufacture of a medicament, preferably a vaccine for the treatment or prevention of KSHV-associated disease or disorder, e.g., Kaposi sarcoma, primary effusion lymphoma (PEL), and multicentric Castleman's disease (MCD).

Another aspect of the disclosure is directed to a composition comprising the peptide or covalently-modified peptide as disclosed above, such as a peptide comprising, consisting essentially of, or consisting of SEQ ID NOs: 1-29 (such as any one of SEQ ID NOs: 1-6 or 1-23), and a pharmaceutically acceptable adjuvant, carrier, or excipient.

In some embodiments a peptide epitope as disclosed herein is complexed with a HLA class 1 or HLA class 2 antigen.

In some embodiments, such a composition may comprise 1, 2, 3, 4, 5, 6, 7, 8, 9, 10 or more of the peptides disclosed herein, or peptides or antigens from other cancers. In such a composition the peptide or covalently-modified peptide may have a length of no more than 10, 15, 20, 25, 30, 35, 40, 45, 50, 55, 60, 65, 70 or >70 contiguous amino acid residues.

Such a composition may comprise 1, 2, 3, 4, 5, 6, 7, 8, 9, 10 or more peptides of SEQ ID NOs: 1-29 (such as any one of SEQ ID NOs: 1-6 or 1-23).

The composition may further comprise an adjuvant or be formulated as a peptide-based vaccine. Thus, a further aspect of the invention relates to an immunogen or vaccine comprising the peptide epitopes of SEQ ID NOs: 1-29 (such as any one of SEQ ID NOs: 1-6 or 1-23) described herein, and, optionally a suitable excipient and/or adjuvant. In one embodiment a KSHV antigen or epitope thereof, such as those comprising a sequence of SEQ ID NOs: 1-29 (such as any one of SEQ ID NOs: 1-6 or 1-23) may be bound to an immunogenic carrier such as BSA, KLH, tetanus toxoid or other immunogenic carrier; or may be incorporated into a liposome.

A liposome may be formulated to contain lipid A, muramyldipeptide or IL-1 as immunomodulators. Types and formulations of liposomes suitable for carriers of immunogens are known in the art and are incorporated by reference to Kaskin, K P, et al., UKR BIOKHIM ZH (59(4):100-107 (1978) and to Chapter 4, Liposomal-based therapeutic carriers for vaccine and gene delivery, M. Rahman, et al., NANOTECHNOLOGY-BASED APPROACHES FOR TARGETING AND DELIVERY OF DRUGS AND GENES, 2017, Pages 151-166.

In general, a peptide or modified peptide as described herein may be incorporated into a composition. Typically, such a composition will include a pharmaceutically acceptable excipient or carrier and may further contain an adjuvant or other active agent.

The term carrier encompasses any excipient, binder, diluent, filler, salt, buffer, solubilizer, lipid, stabilizer, or other material well known in the art for use in pharmaceutical formulations, for example, for intravenous administration a carrier may be sodium chloride 0.9% or mixtures of normal saline with glucose or mannose. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005, which is incorporated herein by reference in its entirety.

An adjuvant is a pharmacological or agent that modifies the effect of other agents. Adjuvants may be added to the materials disclosed herein, such as peptides, peptide constructs, cells and nucleic acids to boost the humoral or cellular immune responses and produce more intense or longer-lasting immunity, thus minimizing the dose of material needed.

Adjuvants that may be compounded with, or otherwise used along with the unmodified KSHV antigen or epitope thereof, modified peptides, peptide constructs, cells expressing the KSHV antigen or epitope thereof or nucleic acid encoding KSHV antigen or epitope thereof, such as those encoding peptide epitopes comprising SEQ ID NOs: 1-29 (such as any one of SEQ ID NOs: 1-6 or 1-23). Adjuvants include, but are not limited to, inorganic compounds including alum, aluminum hydroxide, aluminum phosphate, calcium phosphate hydroxide; mineral oil or paraffin oil; bacterial products or their immunologically active fractions, such as those derived killed Bordetella pertussis, Mycobacterium bovis, or bacterial toxoids; organics such as squalene; detergents such as Quil A, saponins such as Quillaja, soybean or polygala senega; cytokines such as IL-1, IL-2 or IL-12; Freund's complete adjuvant or Freund's incomplete adjuvant; and food based oils like Adjuvant 65, which is a product based on peanut oil. Those skilled in the medical or immunological arts may select an appropriate adjuvant based on the type of patient and mode of administration of the materials described herein.

For therapeutic purposes, formulations for parenteral administration of a peptide composition can be in the form of aqueous or non-aqueous isotonic sterile injection solutions or suspensions. The term parenteral, as used herein, includes intravenous, intravesical, intraperitoneal, subcutaneous, intramuscular, intralesional, intracranial, intrapulmonal, intracardial, intrasternal, and sublingual injections, or infusion techniques. These solutions and suspensions can be prepared from sterile powders or granules having one or more of the carriers or diluents mentioned for use in the formulations for oral administration, preferably in a digestion-resistant form such as an enteric coating. The active ingredient can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Injectable preparations of the KSHV antigen or epitope thereof described herein, for example, sterile injectable aqueous or oleaginous suspensions can be formulated according to the known art using suitable dispersing or wetting ingredients and suspending ingredients. The sterile injectable preparation can also be a sterile injectable solution or suspension in a non-toxic parenterally acceptable diluent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or diglycerides. In addition, fatty acids, such as oleic acid, find use in the preparation of injectables. Dimethyl acetamide, surfactants including ionic and non-ionic detergents, polyethylene glycols can be used. Mixtures of solvents and wetting ingredients such as those discussed above are also useful.

### 4. Methods of Use

In certain aspects, the present disclosure provides methods for eliciting an immune response in a subject having a disease caused by or associated with Kaposi sarcoma-associated herpesvirus (KSHV), e.g., KSHV-associate cancer, comprising administering to the subject a pharmaceutical composition comprising a KSHV-specific T cell (KST) of the present disclosure.

In some other aspects, the present disclosure provides methods for treating a disease caused by or associated with Kaposi sarcoma-associated herpesvirus (KSHV), e.g., KSHV-associate cancer, comprising administering to the subject a pharmaceutical composition comprising a KSHV-specific T cell (KST) of the present disclosure.

In some embodiments, the disease is Kaposi sarcoma (KS), primary effusion lymphoma (PEL), and/or multicentric Castleman's disease (MCD).

In some embodiments, the subject a human.

In some embodiments, the human is immune compromised.

In some embodiments, the human is HIV-positive, such as a human receiving highly active antiretroviral therapy (HAART) for the treatment of HIV who exhibits CD4 immune reconstitution.

In some embodiments, the human is a transplant recipient, and/or is under treatment by an immunosuppressant (such as one or more glucocorticoids, cytostatics, antibodies, and/or drugs acting on immunophilins).

In some embodiments, the subject is co-infected by KSHV and an additional virus (such as HIV, EBV, HHV6, and/or CMV).

In some embodiment, the KSTs are administered parenterally, for example, by intravenous infusion, intraperitoneal infusion, or other parenteral mode. In some embodiments, KSTs may also be infused or administered to a site of cancer, e.g., intratumorally.

In some embodiments, the subject is HLA-matched with the HLA-type of the KST in the pharmaceutical composition. In some embodiments, the KST is derived from a healthy donor who shares at least one HLA class 1 or HLA class 2 allele or HLA antigen with the subject or the recipient. Alternatively, the KST may be autologously derived from the patient or from frozen or stored bone marrow or cord blood of a patient. In a preferred embodiment, the KST is obtained from a healthy donor who shares at least one HLA class 1 or HLA class 2 antigen with the subject.

In some embodiments, the KST is derived from peripheral blood mononuclear cells (PBMCs) of a donor or subject.

In some embodiments, the KST cell is primed and expanded, or expanded, ex vivo or in vitro after recovering them from a donor. A donor may be the patient or a third party donor, often a genetically close family member.

In some embodiments, the pharmaceutical composition comprises different KST cell populations which each recognize a different epitope of KSHV-antigens. For example, the composition may comprise KST populations that recognize two, three, four or more KSHV antigen epitopes, such as those of vFLIP, vIL-6, vCylcin, vGPRCR vIRF3, or those of of SEQ ID NOs: 1-29 (such as any one of SEQ ID NOs: 1-6 or 1-23).

In some embodiments of this method, the KST cell is derived from a T cell obtained from a T cell bank and are selected to comprise at least one, two, three, four, five, six, seven, eight or more HLA class 1 or HLA class 2 antigens shared by a donor and by the subject.

In some embodiments, the pharmaceutical composition comprising a KST is administered in combination with an adjuvant. The adjuvant may be selected from anti-CD40 antibody, imiquimod, resiquimod, GM-CSF, cyclophosphamide, sunitinib, bevacizumab, interferon-alpha, interferon-beta, CpG oligonucleotides and derivatives, poly-(I:C) and derivatives, RNA, sildenafil, particulate formulations with poly(lactide co-glycolide) (PLG), virosomes, interleukin (IL)-1, IL-2, IL-4, IL-7, IL-12, IL-13, IL-15, IL-21, and IL-23.

With the general aspects of the inventions described above, the following example further illustrates specific (non-limiting) embodiments of the invention.

### Example Expansion of KSHV-Specific T Cells from Virus Naïve Donors Towards the Development of an Allogeneic T Cell Therapy for the Treatment of KSHV-Associated Malignancy

Virus specific T cells (VST) are known to play critical roles against KSHV, but reduced responses are seen in patients with Kaposi sarcoma-associated herpesvirus (KSHV) infection and KSHV-associated malignancy, which likely contribute to disease.

A potential immunotherapeutic option, which has shown success in targeting other viruses, is the adoptive transfer of healthy donor-derived VSTs. Infusion of healthy, donor-derived VSTs has demonstrated therapeutic efficacy for the treatment of other oncogenic virus-associated malignancies such as Epstein-Barr virus (EBV) driven lymphomas.

Here, Applicants have successfully expanded KSHV-specific T cell (KST) products against KSHV cancer-associated antigens from 5 confirmed KSHV and HIV naïve donors, and 2 donors whose KSHV and HIV statuses are unconfirmed but otherwise healthy donors. Successful characterization of epitope specific responses are also reported. Naïve CD45RA selected T cells were then isolated from bulk peripheral blood mononuclear cells (PBMCs) to limit expansion of unfavorable memory CD45RO⁺ T cells in these virus naïve donors. These naïve CD45RA-selected T cells were then expanded using dendritic cells pulsed with overlapping peptide pools from select KSHV oncoproteins: vFLIP, vIL-6, vCyclin, vGPCR, and vIRF-3. These antigen targets are highly expressed in the active infection and during oncogenesis. While vFLIP and vIRF-3 do not have significant similarities to human proteins, homology of the other three antigens to human proteins average between 28-35%. However, data presented herein suggests limited cross-reaction of KSHV-specific T cell targets and endogenous human proteins.

In different iterations, KSTs demonstrated specificity to all 5 KSHV oncoproteins by IFN-γ ELISPOT assay, though the five KST products showed varying specificities for vFLIP, vCyclin, and vIRF-3, as evaluated by IFNγ ELISPOT assay.

Following expansion, immunophenotyping of the products revealed a mean of 95% CD3⁺ cells and greater than 90% effector memory T cells in both CD4⁺ and CD8⁺ populations for 4/6 (66.7%) cell products.

Epitope mapping of KSTs revealed unique epitope specificities in KSHV oncoproteins.

Further, upon stimulation with 15-mers, K514, K624, and K974 KST products elicited polyfunctional antigen-specific activation by production of intracellular IFN-γ and TNFα, and non-specific activation was also reported.

To determine minimal epitopes associated with stimulating cytotoxic T cell responses for K514 and K624 products which showed CD8-restricted responses, 9-mer peptides were synthesized from 15-mer epitope specificities and assessed by IFN-γ ELISPOT assay.

K514 KSTs showed robust IFN-γ responses to 5 9-mer epitopes, and K624 KSTs were mapped to an overlapping set of epitopes with the most robust response being for the 9mer: FPLLAECLF (SEQ ID NO: 1).

Next, it was investigated as to whether online MHC-I prediction tools would coincide with the experimental results. Using the IEDB MHC-I binding prediction tool and donor HLA typing, it was found that experimentally determined minimal epitopes for K514 and K624 KSTs were also predicted to be high affinity binders in silico (top 2%).

Epitope specificities for K929 and K017 KSTs were reported.

Table 2 (FIG. 20) summarized the KSHV epitope specificities identified in the KST products and predicted HLA associations based on 15-mer epitopes and donor HLA. Here, it was also show that KSHV-specific T cells can be expanded against v-IL6, a protein that is overexpressed in KSHV⁺ lymphoproliferative disorders, MCD and PEL (4).

Donor 031120 KSTs showed specific responses for v-IL6 post stimulation 3, with no specific responses in the CD45RA-negative fraction except for the lectin-stimulated positive control. Epitope mapping of the products revealed a pair of overlapping epitope specific responses.

Interestingly, one donor (P001) recognized KSHV antigens in the CD45RA-negative fraction. Post stimulation 2 ELISPOT results were indicative of v-GPCR specificity, however cells only and actin controls increased in SFC/105 cells post stimulation 3.

To improve expansion of v-GPCR specific T cells and generate more product, phytohemagglutinin (PHA) blasts were pulsed with v-GPCR peptides alone prior to co-culture with post stimulation 3 P001 KSTs. Post stimulation 4, KSTs had robust IFN-γ production for v-GPCR peptides alone.

Epitope mapping of the products showed specificity for a pair of overlapping peptides, which are homologous to human GPCR.

In silico MHC-I binding prediction was conducted based on the donor HLA. All epitope specificities in the top 2% of binders were reported in the table with HLA-A*02 and HLA-C*02 as the predicted HLA restrictions.

To evaluate cytolytic activity of P001 KSTs, autologous PHA blasts pulsed with actin (control) and v-GPCR pepmixes were used as targets. It was shown that killing of v-GPCR pulsed PHA blasts was higher than the actin condition after 24 hours.

In summary, KSTs were polyfunctional and polyclonal with a predominantly effector memory/effector phenotype (mean CD3⁺ 90%).

Donor K624 products elicited IFNγ and TNFα CD8-restricted responses for overlapping vFLIP peptides, mapped to 9-mer epitope: FPLLAECLF (SEQ ID NO: 1).

Donor K514 products showed CD8 vIRF-3 specificities for epitopes within 2 peptides: LSKGLHPRDLLGSPITAFG (SEQ ID NO: 8) and GLPPASRRRPVVGEFLWDD (SEQ ID NO: 3).

Donor K974 products elicited robust IFNγ and TNFα CD4-restricted responses for vCyclin epitope: SKLRSLTPISTSSLC (SEQ ID NO: 4).

Donor K929 demonstrated robust specificity for vIRF3-LYHGNNPPKKFGVIC (SEQ ID NO: 5) and vCyclin-LDFWHHEVNTLITKA (SEQ ID NO: 6).

KST epitope specificities and predicted HLA associations are shown in FIG. 20. The epitopes sequences are, from top to bottom, SEQ ID NOs: 7-23, respectively.

To our knowledge, KSHV-specific T cell products expanded from virus naïve donors eliciting these antigen-specific responses have not been previously described. This work provides evidence towards the development of an allogeneic, or “off-the-shelf,” KSHV-specific T cell therapy.

In conclusion, these studies support the development of an allogeneic T cell therapy for the treatment of patients with KSHV infection and associated disease, and inform a peptide-based vaccine design.

Materials and methods used in the above example are provided below. It should be understood that the specific conditions used, where themselves constitute specific embodiments of the invention, are not limiting with respect to the general scope of the inventive concept.

### Materials and Methodologies

KSHV peptide libraries. Overlapping libraries of crude 12- to 15-mer peptides encompassing KSHV vFLIP (45 peptides), vIL-6 (49 peptides), vCyclin (62 peptides), vGPCR (83 peptides), and vIRF-3 (139 peptides) were purchased from A&A Labs (San Diego, CA). Peptides were reconstituted in dimethyl sulfoxide (DMSO) (Sigma-Aldrich, St. Louis, MO) at a working concentration of μg/μL.

The publicly available protein sequence used to generate vFLIP peptides is as follows:

(SEQ ID NO: 30)
MATYEVLCEVARKLGTDDREVVLFLLNVFIPQPTLAQLIGALRALKEEG
RLTFPLLAECLFRAGRRDLLRDLLHLDPRFLERHLAGTMSYFSPYQLTV
LHVDGELCARDIRSLIFLSKDTIGSRSTPQTFLHWVYCMENLDLLGPTD
VDALMSMLRSLSRVDLQRQVQTLMGLHLSGPSHSQHYRHTP.

The publicly available protein sequence used to generate vIL-6 peptides is as follows:

(SEQ ID NO: 31)
MCWFKLWSLLLVGSLLVSGTRGKLPDAPEFEKDLLIQRLNWMLWVIDEC
FRDLCYRTGICKGILEPAAIFHLKLPAINDTDHCGLIGFNETSCLKKLA
DGFFEFEVLFKFLTTEFGKSVINVDVMELLTKTLGWDIQEELNKLTKTH
YSPPKFDRGLLGRLQGLKYWVRHFASFYVLSAMEKFAGQAVRVLDSIPD
VTPDVHDK.

The publicly available protein sequence used to generate vCYCLIN peptides is as follows:

(SEQ ID NO: 32)
MATANNPPSGLLDPTLCEDRIFYNILEIEPRFLTSDSVFGTFQQSLTSH
MRKLLGTWMFSVCQEYNLEPNVVALALNLLDRLLLIKQVSKEHFQKTGS
ACLLVASKLRSLTPISTSSLCYAAADSFSRQELIDQEKELLEKLAWRTE
AVLATDVTSFLLLKLLGGSQHLDFWHHEVNTLITKALVDPKTGSLPASI
ISAAGCALLVPANVIPQDTHSGGVVPQLASILGCDVSVLQAAVEQILTS
VSDFDLRILDSY.

The publicly available protein sequence used to generate vGPCR peptides is as follows:

(SEQ ID NO: 33)
MAAEDFLTIFLDDDESWNETLNMSGYDYSGNFSLEVSVCEMTTVVPYTW
NVGILSLIFLINVLGNGLVTYIFCKHRSRAGAIDILLLGICLNSLCLSI
SLLAEVLMFLFPNIISTGLCRLEIFFYYLYVYLDIFSVVCVSLVRYLLV
AYSTRSWPKKQSLGWVLTSAALLIALVLSGDACRHRSRVVDPVSKQAMC
YENAGNMTADWRLHVRTVSVTAGFLLPLALLILFYALTWCVVRRTKLQA
RRKVRGVIVAVVLLFFVFCFPYHVLNLLDTLLRRRWIRDSCYTRGLINV
GLAVTSLLQALYSAVVPLIYSCLGSLFRQRMYGLFQSLRQSFMSGATT.

The publicly available protein sequence used to generate vIRF-3 peptides is as follows:

(SEQ ID NO: 34)
MAGRRLTWISEFIVGALDSDKYPLVKWLDRSTGTFLAPAARNDVIPLDS
LQFFIDFKRECLSKGLHPRDLLGSPITAFGKICTTSRRLRRLPGEEYEV
VQGINCRRWRLLCAEVKECWWCVHARTHLHSGSSLWEILYQHSVRLEKH
RRRPRPFVGENSDSSEEDHPAFCDVPVTQTGAESEDSGDEGPSTRHSAS
GVQPVDDANADSPGSGDEGPSTRHSDSQPPPADETTVHTDNVEDDLTLL
DKESACALMYHVGQEMDMLMRAMCDEDLFDLLGIPEDVIATSQPGGDTD
ASGVVTEGSIAASAVGAGVEDVYLAGALEAQNVAGEYVLEISDEEVDDG
AGLPPASRRRPVVGEFLWDDGPRRHERPTTRRIRHRKLRSAYYRVARPP
VMITDRLGVEVFYFGRPAMSLEVERKVFILCSQNPLADISHSCLHSRKG
LRVLLPKPDDNNTGPGDVNLLAAVLRSFASGLVIVSLRSGIYVKNLCKS
TVLYHGNNPPKKFGVICGLSSRAVLDVFNVAQYRIQGHEHIKKTTVFIG
GDPTSAEQFDMVPLVIKLRLRSVTCDD.

Isolation of peripheral blood mononuclear cells (PBMCs). Bulk peripheral blood mononuclear cells (PBMCs) used in T cell product generation were isolated from KSHV-negative donor buffy coats (Gulf Coast Regional Blood Center, Houston, TX). First, each donor buffy coat (50 mL) was diluted 1:2 in 1× DPBS (Life Technologies Corporation, Grand Island, NY). SepMate™ PBMC Isolation Tubes (StemCell Technologies, Cambridge, MA) were filled with 15 mL of Lymphoprep™ (StemCell Technologies), a density gradient medium, and 25 mL of diluted blood was slowly layered onto the Lymphoprep™ in various isolation tubes. The tubes were centrifuged for 15 minutes at 1200×g with acceleration/deacceleration on 9 (Beckham Coulter Life Sciences, Indianapolis, IN). Following the density centrifugation, the PBMCs and plasma layers were poured into fresh 50 mL conical tubes. The PBMCs and plasma mixture was centrifuged for 10 minutes at 400×g, resuspended in 1× DPBS, and an aliquot was collected for counting by automated cell counter.

Isolation of plasma for anti-KSHV IgG ELISA. Prior to diluting donor buffy coats with 1× DPBS, 10 mL of blood was aliquoted for plasma isolation in a 15 mL conical tube. The tube was centrifuged at 1500×g for 15 minutes on acceleration 9 and deacceleration 8. The plasma layer was collected and frozen in 1 mL aliquots, and the remaining mixture was subjected to the ficoll-based density centrifugation process. HHV-8 IgG antibody ELISA kits (MyBioSource Inc., San Diego, CA; Cat #MBS2800428) were used to measure anti-KSHV antibodies according to the manufacturer protocol. Sample wells were compared to positive and negative controls included with the kit. All conditions were plated in duplicate. The plate was read within 5 minutes of adding the stop solution at 450 nm. Per the protocol, negative control optical density values were no more than 0.15, and positive control optical density values were no less than 0.60. For plasma sample wells, a positive was defined as: ODₛₐₘₚₗₑ≥OD_{negative} 0.10. A negative was defined as: ODₛₐₘₚₗₑ<OD_{negative}+0.10.

Generation of monocyte-derived dendritic cells (DCs). PBMCs were incubated with human CD14 MicroBeads (Miltenyi Biotec) in MACS buffer for 20 minutes at room temperature with gentle agitation every 5 minutes as recommended in the manufacturer's protocol. PBMCs were incubated with 80 μL of MACS buffer (500 mL 1× DPBS, 0.5% bovine serum albumin, and 2 mM EDTA) per 10⁷ total cells and 10 μL of CD14 MicroBeads per 10⁶ total cells. PBMCs were washed in 10 mL 1×DPBS and centrifuged for 5 minutes at 400×g. MACS LS columns (Miltenyi Biotec) were placed in the magnetic field of the MACS Separator (Miltenyi Biotec) and rinsed with 3 mL of MACS buffer. PBMCs were resuspended in 3 mL MACS buffer and applied onto the column. The unlabeled cells and the column effluent from washes (2×) with 4 mL MACS buffer were collected in a 15 mL conical tube. CD14⁺ cells were flushed out of the column with 5 mL MACS buffer into a fresh 15 mL conical tube, centrifuged for 5 minutes at 400×g, and resuspended in DC media (CellGenix, Freiburg, Germany) supplemented with 2 mM of GlutaMAX (Life Technologies Corporation). CD14^{+/-} cell fractions were counted by automated cell counter. CD14-negative cells were frozen for later use with matured DCs.

CD14⁺ cells were plated at a density of 1.5×10⁶ cells/well in 2 mL of DC media with GM-CSF (1000 U/mL) and IL-4 (1200 U/mL) in a 12-well plate and maintained at 37° C. in a humidified CO₂ incubator. On day 4, 1 mL DC media was carefully removed from each well of immature DCs and the cells were supplemented with 1 mL DC media with GM-CSF (1000 U/mL) and IL-4 (1200 U/mL). On day 6, DC maturation cytokines were added in 1 mL of DC media to each well as follows: GM-CSF (1000 U/mL), IL-4 (1200 U/mL), LPS (30 ng/mL), IL-6 (100 ng/mL), IL-1β(10 ng/mL), TNFα (10 ng/mL), IFNγ (100 U/mL), and prostaglandin E2 (PGE2) (250 ng/mL) (LPS sourced from Sigma-Aldrich, St. Louis, MO; all others sourced from R&D Systems, Minneapolis, MN). On day 7, between 18-24 hours post maturation, DCs were harvested by careful washing of wells and centrifuged for 5 minutes at 400×g. DC media was aspirated, and the cells were resuspended in 1 mL CTL media (45% RPMI, 45% Click's Medium, 10% human serum, and 2 mM GlutaMAX) and incubated with KSHV peptides (1 μg/each: vFLIP, vIL-6, vCyclin, vGPCR, vIRF3) for 90 minutes at 37° C. and 5% CO₂. Post peptide-pulse, DCs were irradiated at 25 Gy for one cycle, counted, and resuspended at 2.5×10⁵ cells/mL. DCs were generated using this methodology for three T cell stimulations per donor-derived KSHV-specific T cell product.

Generation of KSHV-specific T cells. To generate KSHV-specific T cells, CD45RA⁺ naïve T cells were stimulated with KSHV peptide-pulsed DCs over the course of three stimulations supplemented with cytokines as previously described. On the day of stimulation 1 during the DC-peptide incubation period, CD14-negative cells were thawed and subjected to CD45RA-selection using human CD45RA MicroBeads (Miltenyi Biotec). CD14-negative cells were resuspended in 80 μL of buffer per 10⁷ total cells and 10 μL of CD45RA MicroBeads per 10⁷ total cells, and incubated at room temperature for 30 minutes. The cells were washed in 10 mL 1× DPBS, centrifuged for 5 minutes at 400×g, and applied to the prepped MACS LS columns as previously described for CD14-selection. The labelled cells were flushed from the column, pelleted, resuspended in CTL media and counted. The CD45RA⁺ cells were resuspended at 1×10⁶ cells/mL and combined with 1 mL of matured KSHV peptide-pulsed DCs (2.5×10⁵ cells/mL) in a 4:1 ratio of T cells:DCs in 24-well plates. IL-21 (60 ng/mL; R&D Systems) was added to the cells to promote effector functions in naïve T cells. T cells were kept in culture for 9 days and supplemented with IL-7 (5 ng/mL) and IL-15 (5 ng/mL) on days 3, 6, and 8. On the day of stimulation 2, T cells were harvested, counted, and excess T cells not used for stimulation 2 were frozen. T cells and matured DCs were combined in a 4:1 ratio (1×10⁶ CTLs:2.5×10⁵ DCs) and supplemented with IL-2 (200 U/mL; R&D Systems, Minneapolis, MN). IL-15 (10 ng/mL) was added to the stim 2 cells on days 3 and 5 of culture. Similarly for stimulation 3, T cells and matured DCs were combined in a 4:1 ratio (1×10⁶ CTLs:2.5×10⁵ DCs), supplemented with IL-2 (200 U/mL) and IL-15 (10 ng/mL) feeds on days 3 and 5 of culture. Stimulations 2 and 3 were kept in culture for 7 days. Cultures were split when >70% confluent. Following the three stimulations, T cell products were harvested and cryopreserved for further study.

Generation of phytohemagglutinin (PHA) blasts. PBMCs were cultured with PHA (5 μg/10⁶ cells; Roche Cat #: 11249738001, or Sigma Cat #: L4144) and IL-2 (200U/10⁶ cells; Prometheus, San Diego, CA) for 5 days. On day of stimulation, PHA blasts were pulsed with KSHV peptides for 90 minutes at 37° C. and irradiated at 75Gy. PHA blasts were plated 1:1 PHA blasts to T cells.

HIV-1 p24 ELISA. HIV-1 p24 levels were measured in cell supernatants after 4 days in culture for all donor-derived KSHV-specific T cell products. HIV-1 Gag p24 DuoSet ELISA (Cat #DY7360-05) and DuoSet ELISA Ancillary Reagent Kit 2 (Cat #DY008B) (R&D Systems, Minneapolis, MN) were used according to the manufacturer protocol. The positive control donor sample was infected with HIV SF-162 via virus-cell co-culture for 2 hrs at 37° C. These infected cells were then washed 4 times with 1×PBS, plated at 1×10⁵ cells/mL in a complete medium (RPMI, 10% FBS) with 100 U/mL IL-2, and cultured for 4 days before supernatant was collected for ELISA assay.

IFNγ enzyme-linked immunospot (ELISpot) assays. Cells were plated on anti-human IFNγ monoclonal antibody (mAb) (capture mAb, 1-DIK-purified, Mabtech, Sweden) coated 96-well polyvinylidene fluoride (PVDF) membrane plates (Millipore, Burlington, MA) at 1×10⁵ cells/well in duplicate to evaluate cytokine secretion in response to KSHV antigens, phytohemagglutinin (PHA) or staphylococcal enterotoxin B (SEB) as positive controls, actin as an irrelevant/negative antigen control, and cells without any peptides as a negative control. Assay was conducted as previously described (12). Pooled peptide mapping was used to determine epitope specificities of KSHV-specific T cell products. Briefly, each antigen was divided into pools consisting of 10-15 peptides wherein each peptide was present in 2 pools. IFNγ-specific responses in overlapping pools allowed for identification of individual 15-mers and subsequent confirmation of 15-mer epitopes was also tested by ELISpot assay. Plates were sent for IFN-γ spot-forming cells counting (Zellnet Consulting, Fort Lee, NJ).

Immunophenotyping of T cell products. Staining of cell surface markers on KSHV-specific T cells was performed with CD3-PeCy7, CD4-BV605, CD8-BV421, CD45RO-APC, and CCR7-FITC (Biolegend, San Diego, CA). The flow cytometry results were analyzed using FlowJo™ v10.8 Software (BD Life Sciences, Ashland, OR).

Intracellular cytokine staining. Samples were incubated with FastImmune (BD Biosciences) and KSHV peptides for 1 hour to stimulate intracellular cytokine secretion. Following 1 hour, brefeldin A (1/100; GolgiPlug; BD Biosciences) was added to stimulated cells and incubated for 4 hours prior to staining. Unstimulated and SEB-stimulated cells were plated as negative and positive controls for IFN-γ and TNF-α production. Staining procedure was conducted as previously described (13). Unstained cells and unstimulated stained cells were used as controls. The flow cytometry results were analyzed using FlowJo™ v10.8 Software (BD Life Sciences, Ashland, OR).

HLA typing. Human leukocyte antigen (HLA) class I (HLA-A, —B, -C) and II (HLA-DR, -DQ, -DP) typing covering 11 HLA loci at 4-field resolution were determined by The Sequencing Center (Fort Collins, CO) for each participant.

Chromium-51 release cytotoxicity assay. Cytotoxicity assay was performed and analyzed as previously described (14).

Statistical analyses. Results were evaluated using descriptive statistics (means and ranges). Analysis was performed in GraphPad Prism (GraphPad).

### REFERENCES

1. F. Bihl et al., Kaposi's sarcoma-associated herpesvirus-specific immune reconstitution and antiviral effect of combined HAART/chemotherapy in HIV Clade C-infected individuals with Kaposi's sarcoma. AIDS 21, 1245-1252 (2007).
2. A. Papadopoulou et al., Activity of broad-spectrum T cells as treatment for AdV, EBV, CMV, BKV, and HHV6 infections after HSCT. Science Translational Medicine 6 (2014).
3. I. Tzannou et al., Off-the-shelf virus-specific T cells to treat BK virus, human herpesvirus 6, cytomegalovirus, Epstein-Barr virus, and adenovirus infections after allogeneic hematopoietic stem-cell transplantation. Journal of Clinical Oncology 35, 3547-3557 (2017).
4. S. Sakakibara, G. Tosato, Viral interleukin-6: role in Kaposi's sarcoma-associated herpesvirus: associated malignancies. J Interferon Cytokine Res 31, 791-801 (2011).
5. A. Nalwoga et al., Kaposi's sarcoma-associated herpesvirus T cell responses in HIV seronegative individuals from rural Uganda. Nat Commun 12, 7323 (2021).
6. R. Roshan et al., T-cell responses to KSHV infection: a systematic approach. Oncotarget 8, 109402-109416 (2017).
7. M. Lambert et al., Differences in the frequency and function of HHV8-specific CD8 T cells between asymptomatic HHV8 infection and Kaposi sarcoma. Blood 108, 3871-3880 (2006).
8. J. Stebbing et al., Kaposi's sarcoma-associated herpesvirus cytotoxic T lymphocytes recognize and target Darwinian positively selected autologous K1 epitopes. J Virol 77, 4306-4314 (2003).
9. R. C. Robey et al., The CD8 and CD4 T-cell response against Kaposi's sarcoma-associated herpesvirus is skewed towards early and late lytic antigens. PLoS One 4, e5890 (2009).
10. T. B. Stuge et al., Diversity and recognition efficiency of T cell responses to cancer. PLoS Med 1, e28 (2004).
11. I. Tzannou et al., Off-the-Shelf Virus-Specific T Cells to Treat BK Virus, Human Herpesvirus 6, Cytomegalovirus, Epstein-Barr Virus, and Adenovirus Infections After Allogeneic Hematopoietic Stem-Cell Transplantation. J Clin Oncol 35, 3547-3557 (2017).
12. H. Dave et al., Toward a Rapid Production of Multivirus-Specific T Cells Targeting BKV, Adenovirus, CMV, and EBV from Umbilical Cord Blood. Mol Ther Methods Clin Dev 5, 13-21 (2017).
13. S. Patel et al., HIV-Specific T Cells Can Be Generated against Non-escaped T Cell Epitopes with a GMP-Compliant Manufacturing Platform. Mol Ther Methods Clin Dev 16, 11-20 (2020).
14. G. Weber et al., Generation of tumor antigen-specific T cell lines from pediatric patients with acute lymphoblastic leukemia—implications for immunotherapy. Clin Cancer Res 19, 5079-5091 (2013).

All references cited herein are incorporated herein by reference, preferably at the place of citation for each cited reference.

1. A pharmaceutical composition comprising a Kaposi sarcoma-associated herpesvirus (KSHV)-specific T cell (KST), wherein said KST:
(1) derives from a naïve T cell that has not been previously exposed to KSHV antigens;
(2) has been exposed to, presented with, and/or stimulated by a KSHV antigen or an epitope thereof; and
(3) produces a cytokine produced by activated T lymphocytes, upon exposure to said KSHV antigen or said epitope thereof.

2. The pharmaceutical composition of claim 1, wherein the KST is a human T cell.

3. The pharmaceutical composition of claim 1, wherein said naïve T cell is isolated from PBMC of a subject naïve to KSHV (e.g., not previously infected by/exposed to KSHV), and optionally further naïve to HIV (e.g., not previously infected by/exposed to HIV).

4. The pharmaceutical composition of claim 1, wherein said KSHV antigen comprises a KSHV cancer-associated antigen.

5. The pharmaceutical composition of claim 1, wherein said KSHV antigen comprises vFLIP, vIL-6, vCyclin, vGPCR, vIRF-3, and/or a combination thereof.

6. The pharmaceutical composition of claim 1, wherein said KST has been exposed to, presented with, and/or stimulated by said KSHV antigen or said epitope thereof through contacting a mature antigen-presenting cell (APC, e.g., a mature dendritic cell) that expresses/presents said KSHV antigen or said epitope thereof.

7. The pharmaceutical composition of claim 6, wherein said KST is contacted by said APC in the presence of one or more cytokines conductive to antigen presentation and stimulation of T cells.

8. The pharmaceutical composition of claim 7, wherein said one or more cytokines conductive to antigen presentation and stimulation of T cells comprise IL-2, IL-7, IL-15, IL-21.

9. The pharmaceutical composition of claim 6, wherein said APC is a mature dendritic cell (i) isolated from PBMC as a CD14⁺ immature dendritic cell; (ii) pulsed with said KSHV antigen in the presence of one or more cytokines conductive for dendritic cell maturation (such as GM-CSF, IL-4, IL-6, IL-1β, TNFα, IFNγ, and prostaglandin E2 (PGE2)); and (iii) optionally irradiated (e.g., at 25 Gy).

10. The pharmaceutical composition of claim 1, wherein said epitope thereof comprises any one or more of the epitopes in FIG. 20, and any one more of SEQ ID NOs: 1-6 (e.g., any one or more of SEQ ID NOs: 1-29).

11. The pharmaceutical composition of claim 1, wherein said cytokine produced by activated T lymphocytes comprises IFNγ and/or TNFα.

12. The pharmaceutical composition of claim 1, wherein the naïve T is a CD14⁻CD45RA⁺ naïve T.

13. A method of treating a disease caused by or associated with Kaposi sarcoma-associated herpesvirus (KSHV), in a subject in need thereof, the method comprising administering an effective amount of the pharmaceutical composition of claim 1 to the subject, such that the disease is treated.

14. The method of claim 13, wherein the disease is Kaposi sarcoma (KS), primary effusion lymphoma (PEL), and/or multicentric Castleman's disease (MCD).

15. The method of claim 13, wherein the subject a human.

16. The method of claim 15, wherein the human is immune compromised.

17. The method of claim 16, wherein the human is HIV-positive, such as a human receiving highly active antiretroviral therapy (HAART) for the treatment of HIV who exhibits CD4 immune reconstitution.

18. The method of claim 16, wherein the human is a transplant recipient, and/or is under treatment by an immunosuppressant (such as one or more glucocorticoids, cytostatics, antibodies, and/or drugs acting on immunophilins).

19. The method of claim 13, wherein the subject is co-infected by KSHV and an additional virus (such as HIV, EBV, HHV6, and/or CMV).

20. The method of claim 13, wherein the subject is HLA-matched with the HLA-type of the KST in said pharmaceutical composition.

21. A method of producing a Kaposi sarcoma-associated herpesvirus (KSHV)-specific T cell (KST), the method comprising: contacting a naïve T cell that has not been previously exposed to KSHV antigens with a KSHV antigen or an epitope thereof.

22. The method of claim 21, wherein said KST is a human T cell.

23. The method of claim 21, wherein said naïve T cell is isolated from PBMC of a subject naïve to KSHV (e.g., not previously infected by/exposed to KSHV), and optionally further naïve to HIV (e.g., not previously infected by/exposed to HIV).

24. The method of claim 21, wherein said KSHV antigen comprises a KSHV cancer-associated antigen.

25. The method of claim 21, wherein said KSHV antigen comprises vFLIP, vIL-6, vCyclin, vGPCR, vIRF-3, and/or a combination thereof.

26. The method of claim 21, wherein said naïve T cell is contacted by said KSHV antigen or said epitope thereof through contacting a mature antigen-presenting cell (APC, e.g., a mature dendritic cell) that expresses/presents said KSHV antigen or said epitope thereof.

27. The method of claim 26, wherein said naïve T cell is contacted by said APC in the presence of one or more cytokines conductive to antigen presentation and stimulation of T cells.

28. The method of claim 26, wherein said one or more cytokines conductive to antigen presentation and stimulation of T cells comprise IL-2, IL-7, IL-15, IL-21.

29. The method of claim 26, wherein said APC is a mature dendritic cell (i) isolated from PBMC as a CD14⁺ immature dendritic cell; (ii) pulsed with said KSHV antigen in the presence of one or more cytokines conductive for dendritic cell maturation (such as GM-CSF, IL-4, IL-6, IL-1β, TNFα, IFNγ, and prostaglandin E2 (PGE2)); and (iii) optionally irradiated (e.g., at 25 Gy).

30. The method of claim 21, wherein said epitope thereof comprises any one or more of the epitopes in FIG. 20, and any one more of SEQ ID NOs: 1-6 (e.g., any one or more of SEQ ID NOs: 1-29).

31. The method of claim 21, further comprising verifying production of a cytokine produced by activated T lymphocytes (such as IFNγ and/or TNFα) upon exposing said KST to said KSHV antigen or said epitope thereof.

32. The method of claim 26, wherein the mature dendritic cell has an HLA type that matches said KSHV antigen or said epitope thereof based on FIG. 20.

33. The method of claim 21, wherein the naïve T is a CD14⁻CD45RA⁺ naïve T.
